SEQUENCE LISTING (47 sequences)

SEQ ID NO: 1 - protein, 5 aa, Artificial Sequence
  This is a synthetic sequence.
VPGVG

SEQ ID NO: 2 - protein, 4 aa, Artificial Sequence
  This is a synthetic sequence.
VPGG

SEQ ID NO: 3 - protein, 4 aa, Artificial Sequence
  This is a synthetic sequence.
GGVP

SEQ ID NO: 4 - protein, 4 aa, Artificial Sequence
  This is a synthetic sequence.
GGFP

SEQ ID NO: 5 - protein, 4 aa, Artificial Sequence
  This is a synthetic sequence.
GGAP

SEQ ID NO: 6 - protein, 5 aa, Artificial Sequence
  This is a synthetic sequence.
GXGXP

SEQ ID NO: 7 - protein, 6 aa, Artificial Sequence
  This is a synthetic sequence.
APGVGV

SEQ ID NO: 8 - protein, 35 aa, Artificial Sequence
  This is a synthetic sequence.
GVGVPGVGFPGEGFPGVGVPGVGFPGFGFPGVGVP

SEQ ID NO: 9 - protein, 35 aa, Artificial Sequence
  This is a synthetic sequence.
GVGVPGVGFPGEGFPGVGVPGVGFPGVGFPGVGVP

SEQ ID NO: 10 - protein, 35 aa, Artificial Sequence
  This is a synthetic sequence.
GVGVPGVGVPGEGVPGVGVPGVGFPGFGFPGVGVP

SEQ ID NO: 11 - protein, 35 aa, Artificial Sequence
  This is a synthetic sequence.
GVGVPGVGFPGEGFPGVGVPGVGVPGVGVPGVGVP

SEQ ID NO: 12 - protein, 35 aa, Artificial Sequence
  This is a synthetic sequence.
GVGVPGVGVPGEGVPGVGVPGVGVPGVGVPGVGVP

SEQ ID NO: 13 - protein, 65 aa, Artificial Sequence
  This is a synthetic sequence.
GVGIPGFGEPGEGFPGVGVPGFGFPGFGIPGVGIPGFGEPGEGFPGVGVPGFGFPGFGIPGVGVP

SEQ ID NO: 14 - protein, 35 aa, Artificial Sequence
  This is a synthetic sequence.
GVGVPGVGFPGKGFPGVGVPGVGFPGFGFPGVGVP

SEQ ID NO: 15 - protein, 35 aa, Artificial Sequence
  This is a synthetic sequence.
GVGVPGVGFPGKGFPGVGVPGVGFPGVGFPGVGVP

SEQ ID NO: 16 - protein, 35 aa, Artificial Sequence
  This is a synthetic sequence.
GVGVPGVGVPGKGVPGVGVPGVGFPGFGFPGVGVP

SEQ ID NO: 17 - protein, 35 aa, Artificial Sequence
  This is a synthetic sequence.
GVGVPGVGFPGKGFPGVGVPGVGVPGVGVPGVGVP

SEQ ID NO: 18 - protein, 35 aa, Artificial Sequence
  This is a synthetic sequence.
GVGVPGVGVPGKGVPGVGVPGVGVPGVGVPGVGVP

SEQ ID NO: 19 - protein, 35 aa, Artificial Sequence
  This is a synthetic sequence.
GVGVPGVGFPGEGFPGVGVPGVGFPGKGVPGVGVP

SEQ ID NO: 20 - protein, 35 aa, Artificial Sequence
  This is a synthetic sequence.
GVGVPGVGFPGEGFPGVGVPGVGVPGKGVPGVGVP

SEQ ID NO: 21 - protein, 5 aa, Artificial Sequence
  This is a synthetic sequence.
VPGXG

SEQ ID NO: 22 - protein, 5 aa, Artificial Sequence
  This is a synthetic sequence.
IPGVG

SEQ ID NO: 23 - protein, 11 aa, Artificial Sequence
  This is a synthetic sequence.
GVGVPXGVGVP

SEQ ID NO: 24 - protein, 5 aa, Artificial Sequence
  This is a synthetic sequence.
GXGXP

SEQ ID NO: 25 - protein, 30 aa, Artificial Sequence
  This is a synthetic sequence.
GVGVPGVGVPGKGVPGVGVPGVGFPGFGFP

SEQ ID NO: 26 - DNA, 66 bp, Artificial Sequence
  This is a synthetic sequence.
gaggatccaggcgttggggtaccgggtgttggcgatccgggtaaaggtgtcccggggttggtgtgc

SEQ ID NO: 27 - DNA, 66 bp, Artificial Sequence
  This is a synthetic sequence.
ctggatccaacgcctgggaatccgaaacccggaaagcctacacccggcacaccaacgcccgggaca

SEQ ID NO: 28 - protein, 10 aa, Artificial Sequence
  This is a synthetic sequence.
GVGVPGYGVP

SEQ ID NO: 29 - protein, 45 aa, Artificial Sequence
  This is a synthetic sequence.
GVGIPGEGIPGVGIPGVGIPGEGIPGVGIPGVGIPGEGIPGVGIP

SEQ ID NO: 30 - protein, 30 aa, Artificial Sequence
  This is a synthetic sequence.
GVGIPGVGIPGEGIPGVGIPGVGIPGVGIP

SEQ ID NO: 31 - protein, 30 aa, Artificial Sequence
  This is a synthetic sequence.
GEGIPGVGIPGEGIPGVGIPGVGIPGVGIP

SEQ ID NO: 32 - protein, 45 aa, Artificial Sequence
  This is a synthetic sequence.
GVGIPGKGIPGVGIPGVGIPGKGIPGVGIPGVGIPGKGIPGVGIP

SEQ ID NO: 33 - protein, 30 aa, Artificial Sequence
  This is a synthetic sequence.
GVGIPGVGIPGKGIPGVGIPGVGIPGVGIP

SEQ ID NO: 34 - protein, 30 aa, Artificial Sequence
  This is a synthetic sequence.
GKGIPGVGIPGKGIPGVGIPGVGIPGVGIP

SEQ ID NO: 35 - protein, 110 aa, Artificial Sequence
  This is a synthetic sequence.
GVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGYGIP

SEQ ID NO: 36 - protein, 110 aa, Artificial Sequence; identical to SEQ ID NO: 35
  This is a synthetic sequence.

SEQ ID NO: 37 - protein, 60 aa, Artificial Sequence
  This is a synthetic sequence.
GVGIPGVGIPGVGIPGVGIPGVGIPGVGIPGVGIPGVGIPGVGIPGVGIPGVGIPGYGIP

SEQ ID NO: 38 - protein, 60 aa, Artificial Sequence; identical to SEQ ID NO: 37
  This is a synthetic sequence.

SEQ ID NO: 39 - protein, 45 aa, Artificial Sequence
  This is a synthetic sequence.
GVGIPGVGIPGVGIPGVGIPGVGIPGVGIPGVGIPGVGIPGYGIP

SEQ ID NO: 40 - protein, 45 aa, Artificial Sequence; identical to SEQ ID NO: 39
  This is a synthetic sequence.

SEQ ID NO: 41 - protein, 30 aa, Artificial Sequence
  This is a synthetic sequence.
GVGIPGVGIPGVGIPGVGIPGVGIPGYGIP

SEQ ID NO: 42 - protein, 30 aa, Artificial Sequence; identical to SEQ ID NO: 41
  This is a synthetic sequence.

SEQ ID NO: 43 - protein, 15 aa, Artificial Sequence
  This is a synthetic sequence.
GVGIPGVGIPGYGIP

SEQ ID NO: 44 - protein, 15 aa, Artificial Sequence
  This is a synthetic sequence.
GVGIPGVGIPGYGIP

SEQ ID NO: 45 - protein, 10 aa, Artificial Sequence
  This is a synthetic sequence.
IPGVGIPGYG

SEQ ID NO: 46 - protein, 10 aa, Artificial Sequence
  This is a synthetic sequence.
IPGVGIPGYG

SEQ ID NO: 47 - protein, 10 aa, Artificial Sequence
  This is a synthetic sequence.
IPGVGIPGYG

(19) United States
(12) Patent Application Publication
(10) Pub. No.: US 2003/0087802 A1
(43) Pub. Date: May 8, 2003

(54) Acoustic absorption polymers and their methods of use

(76) Inventor: Dan W. Urry, Birmingham, AL (US)

Correspondence Address: Cooley Godward LLP, Patent Group, Five Palo Alto Square, 3000 El Camino Real, Palo Alto, CA, 94306-2155, US

(21) Appl. No.: 09/746,371

(22) Filed: Dec. 20, 2000

Related U.S. Application Data
(60) Provisional application No. 60/171,861, filed on Dec. 22, 1999.

(51) Int. Cl. A61K 38/04; C07K 17/00; C07K 16/00; C07K 14/00; C07K 7/00; C07K 5/00; C07K 4/00; C07K 2/00; A61K 38/00; A01N 37/18

(52) U.S. Cl. 514/2; 530/300; 514/17; 514/18; 530/330

(57) ABSTRACT

A method for reducing the acoustical noise, sonar cross-section or radar cross-section of an object, in particular low frequency sonar noise, is provided comprising contacting the object with a polymer that has been optionally modified to include a charged (anionic or cationic) site. In a preferred embodiment, the polymer is a bioelastomer that has been modified to include an anionic site.

### CROSS REFERENCE TO RELATED APPLICATIONS

This application claims the benefit of U.S. Provisional Application No. 60/171,861, filed on Dec. 22, 1999.

### ACKNOWLEDGMENTS

This invention was made with government support under Grant Nos. N00014-89-J-1970 and N00014-98-1-0656 awarded by the Office of Naval Research, and Contract No. N00014-98-C-0279. The US Government has certain rights in the invention as a result of this support.

### TECHNICAL FIELD

The present invention relates to the field of low frequency absorption (<100 kHz). More specifically, the invention relates to methods of engineering polymeric materials for absorption at sonar frequencies. The invention also relates to the engineered polymeric materials themselves and their use in reducing acoustical noise, sonar cross-section and radar cross-section.

### BACKGROUND

Submarine acoustical signatures and sonar cross-sections are critical parameters of undersea warfare. A submarine generates its own acoustical signature. More importantly, a submarine has a sonar cross-section. It has been an on-going goal to reduce the sonar cross-section, with yet a continuing need for improvement. Few materials have been developed that can absorb sound over selected frequency ranges. Current methods involve using twelve inch thick layered polyurethane/filler coatings. These classical elastomers, or rubbers, are reasonable possibilities, but they have a fundamental limitation, in that they are composed of random chain networks. Because of this, the possibilities of tuning or designing for motional modes that could maximize absorption over a targeted frequency range becomes limiting. The frequency band of interest is 40 Hz to 40 kHz, more preferably 200 Hz to 7 kHz range. The random nature of materials such as polyurethane makes them useful absorbers in the mid- to high-frequency range. Unfortunately, however, they do not absorb much below 300-400 Hz, and, in general, they exhibit only a weak acoustic absorption, hence the requirement for very thick coatings.

Accordingly, there are no adequate hull coatings for submarines to reduce sonar cross-section. While submarines have been made quieter, their sonar cross sections need to be more effectively reduced. Therefore, there is a need to reduce submarine detection by sonar by means of developing improved hull coating compositions and methods. In particular, there is a need to develop materials that can demonstrate greater absorption in specific frequency range of interest. There also remains a need to improve effectiveness in reducing acoustical noise emitted from submarines by developing methods and compositions for coating machinery rooms. Of course, a very effective hull coating would also serve to provide absorption of sound from within the ship as well as to reduce the sonar cross-section.

Furthermore, a particularly effective coating would be useful for the hulls of all naval ships for the areas in contact with water to limit detection by submarines and to limit target cross-section to active acoustic torpedoes. In addition, there continues to be a great need to reduce the radar cross-section of all ships.

Additionally, in our noise polluted society there is substantial need for sound filtering whether to protect the hearing of those in the military firing ordnance, or of citizens at the shooting range, or of those in machine shops, near jet engines and otherwise in proximity of sound injurious to hearing. The frequency range for hearing is 10 Hz to 20 kHz. The hearing range overlaps the range of interest in reduction of sonar cross-section. Life long assault and even short intense periods of high intensity sound earlier in life limits effectiveness of hearing, especially as age progresses. Furthermore, there is need for materials suitable for more efficient and effective sound proofing of rooms.

There is also a need for transducers capable of sensing an acoustic wave and converting it to an electrical signal of the same frequency. This requires a material, set in motion by an acoustic wave, that results in a dipole moment change of a related frequency.

The present invention provides compositions and methods for meeting those needs by use of protein-based elastomers, which exhibit entropic elasticity by means of regular, non-random structures.

### SUMMARY OF THE INVENTION

It is an object of the invention to design and prepare polymers capable of acoustic absorption and dielectric relaxations over specifiable frequency ranges and to function under given conditions of temperature and pressure and with adequate material strength.

It is an object of the invention to design polymers having controllable mass and hydrophobicity of the repeat units, and controllable cross-link density, along with anionic or cationic sites capable of forming dissociable ion-pairs with a cation or anion, respectively.

Another object of the invention is to design therapeutic agent delivery systems by designing polymers having anionic or cationic sites capable of forming dissociable ion-pairs with a cation or anion, respectively, where the therapeutic agent is part of the ion-pairing.

It is yet another object of the invention to reduce a submarine's cross-section by covering the hull of the submarine with polymers. It is also an object to reduce the sonar signature or level of acoustical noise emitted by a submarine by coating the machine room of the submarine with polymers.

It is another object of the invention to reduce the damaging effects to both military and civilian personnel of environmental and work-related noise pollution with the use of materials in ear-plugs, ear-muffs, etc. capable of selectively absorbing sound over designated frequency ranges.

It is yet another object to design polymers that are capable of a range of low frequency acoustic absorptions, in the range of 1 Hz to 100 kHz, and that exhibit dielectric relaxations over the same range.

It is still another object of the invention to design materials capable of reducing the radar cross-section of ships with absorptions in the range of 1 MHz to 30 GHz.

It is another object of the invention to tune, by varying the hydrophobic composition of repeating units, controlling the extent of cross-linking and stretching, the low frequency absorptions and the ion-pair dissociation rate of ionic site-containing protein-based polymers in order to extend sound absorption capacity over a desired frequency range and with efficient absorption.

Another object of the invention pertains to a method for reducing the acoustical noise, reducing the sonar cross-section, or reducing the radar cross-section of an object comprising covering the object with a polymer which comprises repeating peptide monomeric units selected from the group consisting of nonapeptide, pentapeptide and tetrapeptide monomeric units of varying mass, wherein said monomeric units form a series of β-turns separated by dynamic bridging segments suspended between said β-turns, wherein each repeating unit exhibits low frequency motional modes. The polymer may optionally be modified to include anionic or cationic sites.

Another object of the invention is a method for reducing the acoustical noise of an object comprising covering the object with an amphiphilic petroleum-based polymer, which exhibits lower critical solution temperature behavior and contains at least one hydrophobically tuned ion-pair dissociable site.

Yet another object of the invention is a method for measuring the sound absorption capabilities of a protein-based material comprising the steps of: (a) forming a test component from a polymer comprising repeating peptide monomeric units selected from the group consisting of nonapeptide, pentapeptide and tetrapeptide monomeric units, wherein said monomeric units form a series of β-turns separated by dynamic bridging segments suspended between said β-turns, wherein the polymer is optionally modified to include a charged site; (b) exposing the test component to a high intensity, low frequency sound; (c) measuring the dielectric constant of the test component; and (d) correlating the measured dielectric increment of the test component to the level of sound that would be absorbed by a protein-based material when exposed to the high intensity, low frequency sound.

Yet another object of the invention pertains to a method of designing polymers that are capable of low frequency acoustic absorption, comprising the steps of: (a) forming a polymer comprised of repeating peptide monomeric units selected from the group consisting of nonapeptide, pentapeptide and tetrapeptide monomeric units, wherein said monomeric units form a series of β-turns separated by dynamic bridging segments suspended between said β-turns; and (b) optionally introducing a charged site, whether anionic or cationic, on said polymer.

Still another object of the invention is the design of petroleum-based polymers capable of lower critical solution temperatures of hydrophobic association with hydrophobically tuned dissociable ion-pairs.

Still another object of the invention pertains to a method of designing polymers that are capable of low frequency acoustic absorption, which comprises forming a polymer comprised of repeating peptide monomeric units selected from the group consisting of nonapeptide, pentapeptide and tetrapeptide monomeric units, wherein said monomeric units form a series of β-turns separated by dynamic bridging segments suspended between said β-turns, wherein said polymer has one or more of the following characteristics: a mean mass of the repeating unit within the range of 300 to 10,000 daltons; a cross-link density of the matrix within the range of one cross-link per 300 dalton to no cross-links between chains (coacervate state); a water content of the matrix or coacervate within the range of 2% to 99% by weight; a hydrophobicity of the repeating unit within the Tₜ range of −200° C. to 90° C.; and a transition temperature within the range of −100° C. to 120° C. Yet another object of the invention is a protein based polymer for use in reducing acoustical noise, reducing sonar cross-section or reducing radar cross-section comprising a polymer having repeating units and at least one of the aforementioned characteristics.

It is a further object of the invention to economically manufacture the protein based polymers that are useful in the methods of the invention, preferably by means of recombinant DNA technology using either expression transformed E. coli and or expression in transgenic plants.

It is yet another embodiment of the invention to utilize bioelastomers in studying the impact of sound bombardment on protein based materials such a marine mammal and other biological tissues.

### DESCRIPTION OF THE FIGURES

FIGS. 1A-1F are depictions of the molecular structure and acoustic function of poly (GVGVP) (SEQ ID NO: 6, where X¹ is V and X² is V). FIG. 1A is a schematic band representation of the β-spiral showing the β-turns as spacers between the turns of the helical spiral. FIG. 1B is the detailed bond representation in stereo pair showing the β-turns and suspended segments between β-turns. FIG. 1C is the spiral axis view showing the space within the β-spiral occupied by water. When viewed in stereo (for cross-eye viewing), in addition to seeing the bond representation (FIG. 1B) in three dimension, the bond representation overlies each of the other representations. FIG. 1D shows the β-turns functioning as acoustic vanes and the suspended segments as entropic molecular springs. Also depicted is an acoustic compressional wave striking a β-turn acoustic vane.

FIGS. 2 and 3 are experimental acoustic absorption data, seen in terms of the loss factor, for two different elastic protein-based polymers, which demonstrate polymers with the capacity, as a function of temperature, to have acoustic absorption maxima directly overlying a most preferred frequency range of 200 Hz to 7000 Hz. FIG. 2 is 20 Mrad cross-linked Polymer II (SEQ ID NO: 6, where X¹ is V and X² is I and n=260) in water. FIG. 3 is 20 Mrad cross-linked Polymer III (SEQ ID NO: 8) in an aqueous calcium chloride medium. Also included in FIGS. 2 and 3 are two classical random chain network elastomers, natural rubber and polyurethane (PAN 10), which show only low broad loss factor curves, as expected for random chain networks. These figures demonstrate the greater capacity of these elastic protein-based polymers to function in the acoustic absorption of interest, and it shows the fundamental difference between random chain network elastomers and elastomers comprised of dynamic regular repeating structural units.

FIG. 4 is a graphical depiction, using three different polymer compositions (Polymer I, SEQ ID NO: 6, where X¹ is V and X² is V and n=251; Polymer II, SEQ ID NO: 6, where X¹ is V and X² is I and n=260; and Polymer III, SEQ ID NO: 8), of 1n frequency of a particular low frequency dielectric relaxation maximum as a function of the mean molecular mass of repeating unit. The linear relationship suggests an inverse dependence of this dielectric relaxation band on mean mass of the repeating unit.

FIG. 5 is a graphical depiction of the ε″/ε″ₘₐₓ values of chemically cross-linked Polymer II as a function of frequency (Hz), over temperatures ranging from 7-50° C. This data demonstrates the capacity to have a dielectric relaxation over the frequency desired for acoustic absorption.

FIG. 6A is a graphical depiction of G′(ω) and G″(ω) values of 20 Mrad cross-linked Polymer III in the contracted state as a function of test frequency in rad/sec, measured in pure water at 5° C. FIGS. 6B-6D are graphical depictions of G′(ω) and G″(ω) values of 14 Mrad cross-linked Polymer III in the swollen state (FIG. 6B), at mid contraction (FIG. 6C), and at full contraction (FIG. 6D) as a function of test frequency in rad/sec.

FIGS. 7A and 7B graphically depict the temperature dependence of the shear modulus as a function of Mrad cross-linking dose for Polymer I (FIG. 7A) and Polymer II (FIG. 7B). This data demonstrates the dependence of shear modulus on cross-link density and the effect of being below and above the temperature of the inverse temperature transition.

FIGS. 8A-8C are graphs depicting the dependence of relaxation frequency on mean mass of the repeating unit using the slope of the curve obtained in FIG. 4, plotted for poly(IPGVG) (SEQ ID NO: 22) and poly[(IPGVG)ₓ(IPGYG)] (SEQ ID NO: 45) (FIG. 8A); poly(IPGVG) (SEQ ID NO: 22) and poly[(IPGVG)ₓ(IPGY{SO₄}G)] (SEQ ID NO: 46) (FIG. 8B); and poly(IPGVG) (SEQ ID NO: 22) and poly[(IPGVG)ₓ(IPGY{SO₄Mg}G)] (SEQ ID NO: 47) (FIG. 8C); where x=0 to 10 in steps of 1.

### DESCRIPTION OF SPECIFIC EMBODIMENTS

When considering acoustic materials suitable for low frequency acoustic absorption, compliant or elastomeric materials present interesting candidates. Certain protein-based polymers can be dominantly entropic elastomers and yet contain dynamic structural regularities that exhibit motional modes. By unique design, these polymers can be designed to exhibit motional modes over interesting frequency ranges.

The instant invention relates to designing polymers, particularly bioelastomers that have utility as acoustic absorbers. Few materials have been developed that can absorb sound at low frequencies in the range of up to 100 kHz. In particular, there are few materials able to absorb sound at sonar frequencies, which are typically within the range of thousands to hundreds of Hertz.

The instant invention provides for materials that can be engineered to absorb sonar frequencies in the range of up to 100 kHz, typically within the frequency range of about 100 Hz to 100 kHz. These materials are preferably elastic protein-based polymers, referred to herein as bioelastomers. These materials have numerous commercial applications, but are of particular interest in reducing the sonar cross-section and the acoustical signature of submarines by coating or otherwise covering the hull with a bioelastomer of the invention. It is estimated that such use of bioelastomers can reduce a submarine's acoustical signature by as much as 25 decibels (“dB”). It is also expected that non-protein based polymers, modified as described herein, will also be well suited as low frequency acoustic absorbers. In general, the polymers may also be used to limit environmental noise pollution whether in the workplace, in the community or at home.

### The Materials

Bioelastic polymers are preferred for use in the invention. Their defined structure allows the polymers to be designed and synthesized with chosen physical properties, rather than having to rely on the less controllable properties of materials prepared from random chain network elastomers. One means of defining bioelastomers is to describe groups of peptide sequences. These materials may or may not contain ionizable amino acid residues (used for purposes described below). Specifically, these materials may be described as containing repeating units of the formula αPρΩG or αPθδ, wherein: P is a peptide-forming residue of L-proline; G is a peptide-forming residue of glycine; α is a peptide-forming residue of L-valine, L-leucine, L-isoleucine, L-phenylalanine, L-alanine or an ionizable peptide-forming residue selected from the group consisting of the residues of L-Glu, L-Asp, L-His, L-Lys, L-Tyr, and other ionizable peptide-forming L-amino acids; ρ is a peptide-forming residue of glycine or a peptide-forming residue of D-Ala, D-Glu, D-Asp, D-His, D-Lys, D-Tyr, or (optionally) other ionizable peptide-forming D-amino acids for the elastic polymeric repeats or any L-amino acid for the plastic forming repeats; Ω is a peptide-forming residue of L-valine, L-leucine, L-isoleucine, L-phenylalanine or (optionally) an ionizable peptide-forming L-amino acid or any other of the naturally occurring amino acid residues; θ is a peptide-forming residue of glycine or a peptide-forming residue of D-Glu, D-Asp, D-His, D-Lys, D-Tyr, or (optionally) another ionizable peptide-forming D-amino acid; and δ is a peptide-forming residue of glycine or a peptide-forming residue of L-Glu, L-Asp, L-His, L-Lys, L-Tyr, or (optionally) another ionizable peptide-forming L-amino acid or any other of the naturally occurring amino acid residues.

Examples of these artificial or synthetic bioelastomers are described, for example, in Urry, et al., U.S. Pat. No. 4,132,746 (VPGVG and variants) (SEQ ID NO:1); Urry, U.S. Pat. Nos. 4,500,700; 4,898,926; 5,527,610; and 5,336,256, all of which describe tetrapeptide and pentapeptide repeats; Urry, U.S. Pat. No. 4,589,882 (cross-linking); Urry, et al., U.S. Pat. No. 4,783,523 (IPGVG and variants) (SEQ ID NO: 22); Urry, et al., U.S. Pat. No. 4,870,055 (inclusion of hexamers); Urry, U.S. Pat. No. 5,064,430 (nonapeptide repeats); Urry, U.S. Pat. No. 5,250,516 (inverse temperature transition); Urry, et al., U.S. Pat. No. 5,854,387 (purification); and Urry, U.S. Pat. No. 5,900,405; all of which are incorporated herein by reference.

Numerous bioelastomers are described in the patents noted above and incorporated herein by reference. Although these patents have not been concerned with acoustic absorbers, they provide considerable guidance on the manufacture of bioelastomers to obtain useful structural features for the uses described herein.

Another way to describe the bioelastomers suitable for use in the methods of the invention is to define them as polymers which comprise repeating elastomeric peptide monomeric units selected from the group consisting of bioelastic tetrapeptides, pentapeptides, and nonapeptides units, which comprise amino acid residues selected from the group consisting of hydrophobic amino acid and glycine residues. These monomeric units form a series of β-turns separated by dynamic bridging segments suspended between the β-turns, i.e., the monomers exist in a conformation having a β-turn of the formula:

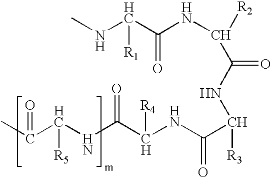

wherein R₁-R₅ represent side chains of amino acid residues 1-5, and m is 0 when the repeating unit is a tetrapeptide or 1 when the repeating unit is a pentapeptide. Nonapeptide repeating units generally consist of sequential tetra- and pentapeptides often with alternating glycines in the tetrapeptide portion. Preferred hydrophobic amino acid residues are selected from the group consisting of alanine, valine, leucine, isoleucine, proline, phenylalanine, tyrosine, tryptophan, and methionine. In many cases, the first amino acid residue of the repeating unit is a residue of valine, leucine, isoleucine, or phenylalanine; the second amino acid residue is a residue of proline; the third amino acid residue is a residue of glycine; and the fourth amino acid residue is glycine or a very hydrophobic residue such as tryptophan, phenylalanine or tyrosine, or any other of the naturally occurring amino acid residues; the fifth amino acid residue is most commonly a glycine.

Bioelastomers can be rationally designed in order to achieve the desired properties appropriate for the methods of the invention. The choice of individual amino acids from which to synthesize the elastomeric units and resulting polypeptide is unrestricted so long as the resulting structure comprises elastomeric structures with features described, for example, in U.S. Pat. Nos. 4,500,700 and 5,064,430, herein incorporated by reference, particularly the β-turn formation described above and the resulting polymer maintains attributes useful for purposes intended according to the embodiments of the invention including, among other things, acoustic absorption.

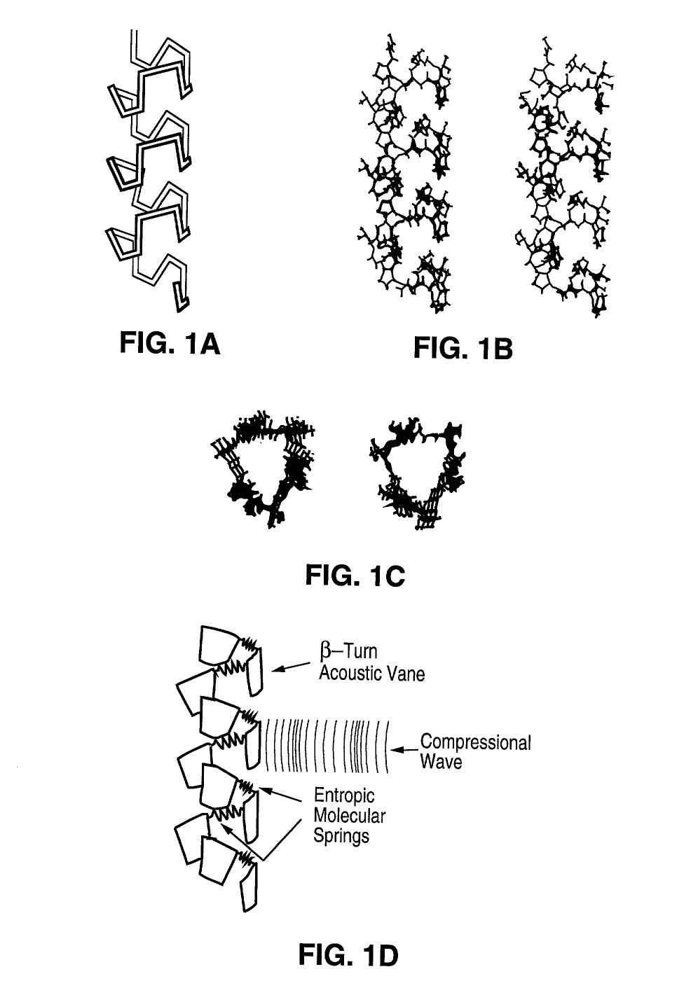

The structure of poly(VPGVG)-type protein-based elastomers can be represented as a helical array of acoustic vanes. As reported by Urry, et al., “Dynamic β-Spirals and A Librational Entropy Mechanism of Elasticity” in Conformation in Biol. (Srinivasan and Sarma, Eds., G. N. Ramachandran Festschrift Volume, Adenine Press, USA, 11-27, 1982), the proposed molecular structure of poly(VPGVG)-type protein-based elastomers is one of a hydrophobically assembled helical array of β-turns connected by dynamic suspended segments. Referred to as a “β-spiral”, this structure is an open helix with water inside. The suspended segments, which connect the β-turns, contain peptide moieties, which exhibit large amplitude rocking (librational) motions that become damped on extension. Thus, the suspended segments that link the β-turns function as entropic molecular springs. The proposed molecular structure and a graphical depiction illustrating the acoustic functional features are shown in FIGS. 1A-1D.

The acoustically relevant features are β-turns arranged on the surface of the helix with their planes parallel to the helix axis. In this structural configuration, the β-turns provide dynamically active surface elements, effectively forming a series of vibrating plates on the surface of the wide, water-containing and dynamic helix. A classical analog would be the helical array of plates connected by springs. The β-turn surface elements may be thought of as molecular acoustic vanes attached to springs that are tunable to resonate over desired frequency ranges.

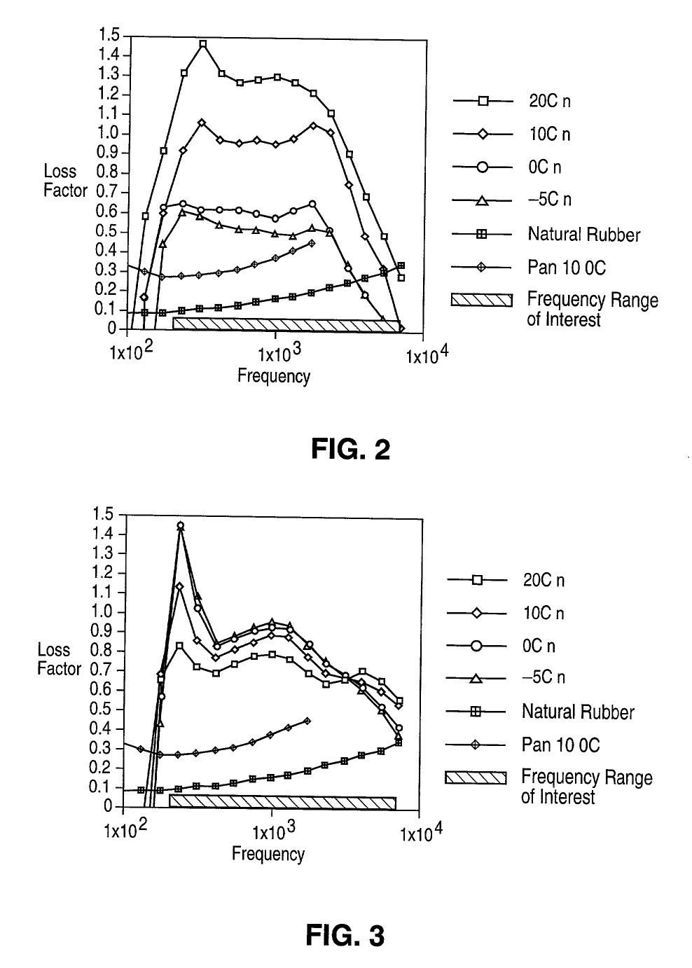

These elastomeric polypeptides have demonstrated acoustic absorption characteristics over the specified frequency range. Because the β-spiral is comprised of repeating structural features, each repeating unit exhibits the same motional elements. Accordingly, the sum of repeated motions along a chain can result in intense frequency-localized motional modes. The situation is quite different for a random chain network. The energy barrier for torsional motion will be different for each repeating bond in a random chain network. Thus, intense, localized relaxations are expected for regular, non-random, elastomers as described in FIGS. 1A-1D, whereas only weak, broad and featureless relaxation spectra are expected for random chain network elastomers, as seen in FIGS. 2 and 3.

In the past it has not been possible to compare, so directly, motional characteristics of elastomeric polypeptides comprised of repeating peptide sequences with those of classical rubbers. This is because the classical rubbers do not generally have significant dipole moments, which could be characterized by dielectric relaxation measurements, as described below. Since acoustic absorption is a mechanical property, not a dielectric property dependent on strong dipole moments, acoustic absorption of different elastic materials can be compared, independent of their chemical composition. This occurs in the comparisons of FIGS. 2 and 3. These figures were arrived at by computational analysis of data for two different sample thicknesses, 0.5 cm and 1.0 cm.

The data of FIGS. 2 and 3 become a classic delineation between random chain network elastomers and elastomers comprised of regular, non-random, albeit dynamic repeating structural elements. Natural rubber and a polyurethane elastomer, both described as random chain networks, exhibit an acoustic absorption spectrum that is of low intensity and broad. On the other hand, the acoustic absorption spectra for elastomeric polypentapeptides exhibit an intense localized absorption band that increases in intensity as more polypeptide folds into its regular structure on raising the temperature, as seen for example in FIG. 2.

The intense acoustic absorption band exactly overlies the frequency range specified for lowering the sonar cross-section of naval ships. Accordingly, it now must be ensured that the desired absorption properties are maintained as a function of the operating temperatures and pressures and that the materials have adequate mechanical properties of stability, compressibility set, creep, and tensile strength. Furthermore, additional mechanism of ion-pair dissociation/association can be introduced to enhance absorption further and to further tune frequency. The background information required for the design of materials that would ensure the above properties is briefly presented below.

However, the methods of the invention are not limited to using protein based polymers such as bioelastomers. Non-protein-based polymers such as petroleum based polymers, modified as described herein, are also suitable for use in one element of the invention. These include by way of illustration and not limitation, petroleum based polymers including acrylamides such as poly(N-isopropyl acrylamide) and poly(N-acryloly-I-valine) and esters such as poly(methylmethacryalic acid) and with larger aliphatic esters, e.g., ethyl, propyl, isopropyl, and with aromatic esters. These are polymers that exhibit a lower critical solution temperature (“LCST”) that is equivalent to the inverse temperature transition of bioelastomers. The structure and properties of such petroleum-based materials can not be engineered to the same extent as is done with protein based polymers. However, many non-protein based polymers have the advantage of being more stable, and hence less biodegradable. Therefore, depending upon the particular use intended, non-protein-based polymers may be preferred over protein-based ones.

Accordingly, as used herein, the term “polymer” is intended to include protein based polymers and non-protein based polymers. It should also be noted that the terms “bioelastic polymers”, “elastic protein-based polymers”, “bioelastomeric polymers” and “bioelastomers” are used interchangeably herein and further that these terms encompass materials that may not be thought of as elastomers (such as certain polymers having the characteristics of plastics), since the term “bioelastomer” has come to be an art-recognized term.

Before describing the considerations in selecting and/or designing a polymer with the appropriate acoustic absorption characteristics, such as hydrophobic characteristics, and the process by which an ionic member is added to the polymer, a basic understanding of the preferred polymers, bioelastomers, is useful.

Bioelastomers can be designed to have numerous advantages, which can be achieved by providing polymers comprised of easily obtained and coupled monomer units, e.g. amino acids, that are themselves diverse in structure and in chemical properties and are readily modified. Thus, the polymer can also be present as a copolymer containing a mixture of tetrameric, pentameric or other monomeric units. Furthermore, recombinant peptide-engineering techniques can be advantageously used to produce specific peptide backbones, either in bioelastic units or non-elastic biofunctional segments.

The polymers can be prepared with widely different water compositions, with a wide range of hydrophobicities, with almost any desired elastic modulus, in numerous different physical forms (e.g., sheets, gels, foams, powders, and so forth), and with a variable degree of cross-linking by selecting different amino acids for the different positions of the monomeric units and by varying the cross-linking process (e.g. chemical, enzymatic, or radiation) used to form the final product. Preparation of a variety of polymers, taking into consideration these numerous aspects of polymer design, has already been described, for example in the patents referenced herein, and will therefore only be briefly described here.

The preferred bioelastomers useful in the methods of the invention are polymers comprising repeating tetrapeptide, pentapeptide and/or nonapeptide monomeric units, i.e., polytetrapeptides, polypentapeptides and polynonapeptides. The polymer may optionally be modified to include an ionic site, preferably an anionic site. Typical bioelastomers useful in this invention contain at least 5, preferably at least 10, more preferably at least 20 monomers, and even more preferably at least 100 monomers. The bioelastomers can also optionally have insertions of, for example, single amino acids between monomeric units, substitutions of one amino acid for another in an occasional monomer, or inclusion of different tetrapeptide, pentapeptide or nonapeptide sequences which can be added either in parallel or in sequence to increase strength in elastic modulus or provide some other desired characteristic. See U.S. Pat. Nos. 4,500,700 and 5,064,430. The resulting polymers are thus properly known as copolymers, as they are formed from different monomeric units. A typical copolymer will preferably be a mixture of tetrapeptide and pentapeptide units, which may be the same or different, i.e., all of the tetramers may be the same or they may be different and all pentamers may be the same or they may be different. In addition, the bioelastomer can be a copolymer formed from one of the aforementioned monomeric units and a second peptide unit containing 1-200 amino acids, typically 1-100 amino acids, and more typically 1-20 amino acids. Such a second peptide may have many uses such as being introduced to modify the elastic modulus, such as the hexamer, -APGVGV- (SEQ ID NO: 7), described in Urry, et al., U.S. Pat. No. 4,870,055.

### Amino Acid Selection

As noted above, there are certain characteristics that are critical to the performance of the polymers in the methods of the invention. However, as is apparent to one of ordinary skill in the art, there are numerous other physical properties of the polymer that can be adjusted to exhibit desired characteristics, for example, viscosity, viscoelasticity, consistency, modulus of elasticity, stability, toughness, water composition, degree of hydrophobicity, physical forms and degree of cross-linking, some of which are described in detail below. Accordingly, selection of the sequence of amino acids in a particular monomeric unit and selection of the required proportion of monomeric units can be accomplished by an empirical process that begins with determining (or looking up) the properties of known bioelastomers, making similar but different bioelastomers and measuring the physical properties as described herein and in the patents referred to above. From there, modifications can be rationally made to the selection process in order to arrive at a bioelastomer having the desired properties.

Tetramers or tetrapeptide monomeric units of particular interest include VPGG (SEQ ID NO: 2), GGVP (SEQ ID NO: 3), GGFP (SEQ ID NO: 4) and GGAP (SEQ ID NO: 5).

Examples of suitable pentamers or pentapeptide monomeric units include, by way of illustration and not limitation, those units describes as GX¹GX²P (SEQ ID NO: 6), where X¹ is selected from the group consisting of valine (V), glutamic acid (E), phenylalanine (F), tyrosine (Y), and lysine (K); while X² is selected from the group consisting of V, E, F and isoleucine (I). Specific examples include, GVGVP (SEQ ID NO: 6, where X¹ is V and X² is V), GVGIP (SEQ ID NO: 6, where X¹ is V and X² is I), GVGFP (SEQ ID NO: 6, where X¹ is V and X² is F), GFGFP (SEQ ID NO: 6, where X¹ is F and X² is F), GFGEP (SEQ ID NO: 6, where X¹ is F and X² is E), GFGIP (SEQ ID NO: 6, where X¹ is F and X² is I), GEGFP (SEQ ID NO: 6, where X¹ is E and X² is F), GEGVP (SEQ ID NO: 6, where X¹ is E and X² is V), GKGFP (SEQ ID NO: 14), GKGVP (SEQ ID NO:6, where X¹ is K and X² is V), GEGIP (SEQ ID NO: 6, where X¹ is E and X² is I), GKGIP (SEQ ID NO: 6, where X¹ is K and X² is I) and GYGIP (SEQ ID NO: 6, where X¹ is Y and X² is I). For other specifically preferred individual monomeric units and bioelastomers, see SEQ ID NOS: 43 through 58 and any of the patents that are herein incorporated by reference.

Particularly preferred bioelastic materials are those that contain at least one GVGVP (SEQ ID NO: 6, where X¹ is V and X² is V) or GVGIP (SEQ ID NO: 6, where X¹ is V and X² is I) pentapeptide, which can also be referred to as a-(GVGVP)ₙ-b or a-(GVGIP)ₙ-b polymers, where “n” is an integer from 1 to 10,000, preferably 3 to 700, and “a” and “b” are polytetrapeptides, polypentapeptides, nonapeptides or copolymers thereof.

### Elasticity

One means of controlling the elastic modulus of polymers pertains to a characteristic referred to as the transition temperature (“Tₜ”). A unique aspect of some bioelastomers is that they undergo an inverse temperature transition, during which a regular structure develops, unlike the random network structure of typical rubbers. This is described in detail in Urry, U.S. Pat. No. 5,250,516. At temperatures above its Tₜ, a bioelastomer associates reversibly to form a dense, water-containing viscoelastic phase, which is called the coacervate, and the solution above the coacervate is referred to as the equilibrium solution. This process of raising the temperature to form the elastomeric state results in the development of a regular structure that is a β-spiral, a loose water-containing helical structure with β-turns as spacers between turns of the helix which provides hydrophobic contacts between helical turns and with suspended peptide segments. Accordingly, the elastomeric force of these bioelastomers develops as the regular structure thereof develops. By synthesizing bioelastic materials having varying molar amounts of the constituent repeating units and by choosing a particular solvent to support the initial viscoelastic phase, it is possible to rigorously control the temperature at which the obtained bioelastomer develops elastomeric force. Maximum elastomeric force develops over a relatively narrow temperature range at temperatures spanning a range of up to about 75° C.

Accordingly, one consideration in selecting the sequence of amino acids in a particular monomeric unit and selection of the required proportion of monomeric units can be accomplished by an empirical process that begins with determining (or looking up) the properties of known bioelastomers, making similar but different bioelastomers and measuring the Tₜ and physical properties as described herein and in the patents referred to above. For example, the effect of changing the amino acid composition on the value of the transition temperature (“Tₜ”) can be determined using a hydrophobicity scale such that a rough estimate of the likely Tₜ can be obtained by summing the mean hydrophobicities of the individual amino acid residues in the monomeric units of the polymer and comparing the result to the sum obtained for polymers having known Tₜ. Typically, more hydrophobic residues (e.g., Ile, Phe) lower Tₜ, whereas less hydrophobic residues (e.g., Ala, Gly) and polar residues (e.g., Asp, Lys) raise Tₜ. Virtually every variable can, with the appropriate composition of the protein-based polymer, change the value of Tₜ. Such variables include, among other criteria, (1) polymer concentration, (2) polymer length, (3) amino acid composition, (4) presence of salts e.g. the Hofineister (Lyotropic) Series, (5) organic solutes and solvents, (6) polymer side-chain ionization, (7) chemical modification of polymer side-chains e.g. phosphorylation, sulfation or nitration, (8) pressure e.g., as effecting aromatic residues, (9) redox state of chemical groups attached to the polymer, (10) light absorption by chemical groups attached to the polymer, and (11) side chain neutralization by ion pairing e.g., cation neutralization of anionic side chains, anion neutralization of cationic side chains, and ion-pairing between side chains.

### Backbone Modifications

The bioelastic polymers are composed of peptide units that form a matrix, which can be modified in a variety of ways to obtain additional properties. For example, one or more of the peptide bonds can be optionally replaced by substitute linkages such as those obtained by reduction or elimination. Thus, one or more of the —CONH— peptide linkages can be replaced with other types of linkages such as —CH₂NH—, —CH₂S—, CH₂CH—, —CH═CH— (cis and trans), —COCH₂—, —CH(OH)CH₂—, and —CH₂SO—, by methods known in the art, for example, see Spatola, A. F. (1983) in “Chemistry and Biochemistry of Amino Acids, Peptides and Proteins” (B. Weinstein, ed., Marcel Dekker, New York), p. 267 for a general review. Amino acid residues are preferred constituents of these polymer backbones. Of course, if backbone modification is made in the elastomeric units, then suitable backbone modifications are those in which the elasticity and inverse temperature transition of the polymer is maintained.

### Cross-Linking

The degree of cross-linking can be controlled by selecting different amino acids for the different positions of the monomeric units and by varying the cross-linking process (e.g. chemical, enzymatic, or radiation) used to form the final product. For example, polymer characteristics can be affected by cross-linking using any of various cross-linking processes, e.g., chemical, enzymatic, or irradiative. Cross-linking provides mechanical strength and rigidity to the polymer, and increasing amounts of cross-linking are appropriate for increasing demands of rigidity. Cross-linking to provide one cross-link for every 200-500 repeating units is generally acceptable, with more cross-linking being permitted in less viscous polymers and vice versa. Methods for cross-linking bioelastomeric polypeptides are known in the art, such as Urry, U.S. Pat. No. 4,589,882, which teaches enzymatic cross-linking by synthesizing block polymers having enzymatically cross-linkable units. For example, cysteine can be introduced into the polymer to allow for linkage via disulfide bridges to a surface, or lysine can be introduced for enzymatic linkage to a surface, using an enzyme that cross-links for example, collagens and elastins. Another example is a bioelastomer containing one or more monomers that have a lysine (K) residue, such as GKGVP (SEQ ID NO: 6, where X¹ is K and X² is V), which has been shown to be a substrate for the cross-linking enzyme lysyl oxidase.

Cross-linking may also be achieved by use of water soluble carbodiimides to cross-link the carboxyls of glutamic acid (Glu, E) or aspartic acid (Asp, D) on one chain to the amino function of a lysine (Lys, K) residue of another chain to form an amide. This is relevant to the carboxyl or Glu-containing sequences (SEQ ID NOS: 19, 20, 21, 22, and 23) combining with the amino or Lys-containing sequences (SEQ ID NOS: 25, 26, 27, 28 and 29), and to the carboxyl or Glu-containing sequences (SEQ ID NOS: 43, 44, and 45) combining with the amino or Lys-containing sequences (SEQ ID NOS: 46, 47 and 48). The approach to this chemical cross-linking using water soluble carbodiimide is the following: A solution of Glu-containing polymer in water (40 mg/mL) at pH 7.5 is mixed with a solution of Lys-containing polymer in water (40 mg/mL) at pH 7.5. The solution is equilibrated at 2 to 3° C. above its transition temperature. A calculated quantity of EDCI and HOBt is added. The pH is adjusted to 7.5 with N-methylmorpholine and maintained above temperature with shaking for two days.

This invention also contemplates cross-linking the bioelastomer by first contacting the object of interest with the bioelastomer, for example by coating its surface with a viscous bioelastomer composition, followed by coating its surface with a cross-linking agent.

Such cross-linking agents include, by way of illustration and not limitation, cerric iron and sodium hyposulfite. Attachment of polymer to polymer or of polymer to surface can also occur using the SH functional group of cysteine (Cys, C).

Additionally, cross-linking by irradiation is described in detail in nearly all of the patents referred to herein. For example, (GVGVP)ₙ(SEQ ID NO:6, where X¹ is V and X² is V), when prepared with n on the order of 200 and when cross-linked with 20 Mrads of γ-irradiation, forms an elastic matrix with an elastic modulus in the range of 10⁵ N/m². By variations in composition and conditions, the elastic modulus can be varied from 10⁴ to 10⁸ N/m². Another such polymer is X²⁰-poly(GVGVP) (Urry, et al., J. Bioactive Compatible Polym. 6:263-282 (1991)). Polymers that are prepared by irradiation cross-linking are identified as, for example, “X²⁰-polyVPGVG,” which refers to a polymer prepared from VPGVG pentapeptide units which has been irradiated with a 20 Mrad dose of cobalt-60 radiation to form the cross-links, thus resulting in an insoluble matrix. Cross-linked coacervates can also be obtained by much higher and lower radiation dosages, as high as 50 Mrad, but usually less than 20 Mrad, often less than 10 Mrad, and even less than 5 Mrad.

### Overall Amino Acid Composition

Considerable variations in the amino acids that are present at various locations in the resulting polymer is also possible as long as the multiple β-turns with intervening suspended bridging segments are retained in order to preserve elasticity. For this reason, it is preferred that at least 50% of the polypeptide is formed from the repeating monomeric units, more preferably at least 70%, even more preferably at least 90%. Nevertheless, it is possible to prepare polypeptides in which these monomeric units are interspersed throughout a larger polypeptide that contains peptide segments designed for other purposes. Such sequences can be added covalently and sequentially or as side chains to provide for the desired function. The ratio of these other sequences to the monomer residue can range from 1:2 to 1:5000. Preferably the ratio is 1:10 to 1:100. The upper limit on the number and kind of substituents is also influenced by the ability of the elastic polymer to fold/assemble properly to attain a beta-spiral in the relaxed state.

### Overall Hydrophobicity

The hydrophobicity of the overall polymer (and therefore the average hydrophobicity of functional groups present in the polymer) can be modified by changing the ratio of different types of monomeric units. These can be monomeric units containing a functional group undergoing the transition or other monomeric units present in the polymer. For example, if the basic monomeric unit is GVGVP (SEQ ID NO: 6, where X¹ is V and X² is V) and the unit undergoing transition is GX⁴GVP (SEQ ID NO: 21), where X⁴ is an amino acid residue modified to have an electroresponsive side chain, either the ratio of GVGVP unit to GX⁴GVP units can be varied or a different structural unit, such as GVGIP (SEQ ID NO: 22), can be included in varied amounts until the appropriate transitions temperature is achieved. Furthermore the precisely specifiable sequence of structure of the protein and protein-based bioelastic polymers allows optimal arrangement of the structural components. For example, optimal spatial proximity can be achieved by placing coupled residues adjacent to each other in the backbone (i.e., based on primary sequence) and also by positioning to provide inter-turn proximity.

A major advantage of the bioelastic polypeptides is the extent to which fine-tuning of the degree of hydrophobicity/polarity and resulting shift in the inverse temperature transition can be achieved. In addition to changes to the amino acid composition as noted above, any chemical means of changing the mean hydrophobicity of the polymer, such as dephosphorylation and phosphorylation, reduction and oxidation of a redox couple, ionization and deionization, protonation and deprotonation, cleavage and ligation, amidation and deamidation, a conformational or a configurational change (e.g., cis-trans isomerization), an electrochemical change (e.g., pKa shift or reduction potential shift), emission/absorbance, or other physical change (e.g., heat energy radiation/absorbance), pressure (See U.S. Pat. No. 5,226,292), photoresponsive or electroresponsive effects, or combinations thereof, can be used to change the Tₜ.

The hydrophobicity is easily designed by selection of appropriate amino acid residues. There is a discussion of this selection process below, as it pertains to hydrophobic characteristics of bioelastomers in general. For their specific use in the methods of the instant invention, there are three preferred residues that occur in one or more of the monomeric units making up the bioelastomer: phenylalanine, tyrosine and isoleucine. Accordingly, in one preferred embodiment of the invention, the polymer comprises at least one monomeric unit containing a phenylalanine or isoleucine residue. Pentamers of particular interest are charged and hydrophobically varied analogues of bioelastomers based upon the GVGVP (SEQ ID NO: 6, where X¹ is V and X² is V) pentapeptide and GVGIP (SEQ ID NO: 6, where X¹ is V and X² is I). Accordingly, in one embodiment of the invention preferred bioelastomers contain at least one such pentamer analogue having the formula GX¹GX²P (SEQ ID NO: 6), where X¹ is selected from the group consisting of valine (V), glutamic acid (E), phenylalanine (F), tyrosine (Y), and lysine (K); while X² is selected from the group consisting of V, E, F and isoleucine (I).

Preferred X¹ residues include F, such as in the pentamers GFGFP (SEQ ID NO: 6, where X¹ is F and X² is F), GFGEP (SEQ ID NO: 6, where X¹ is F and X² is E), and GFGIP (SEQ ID NO: 6, where X¹ is F and X² is I), Y such as in the pentamer GYGIP (SEQ ID NO: 6, where X¹ is Y and X² is I), E such as in the pentamer GEGIP (SEQ ID NO: 6, where X¹ is E and X² is I) and K such as in the pentamer GKGIP (SEQ ID NO: 6, where X¹ is K and X² is I), for example. A preferred X² residue is selected from the group consisting of F and I, such as in the pentamers GVGIP (SEQ ID NO: 6, where X¹ is V and X² is I), GFGIP (SEQ ID NO: 6, where X¹ is F and X² is I), GVGFP (SEQ ID NO: 6, where X¹ is V and X² is F), GFGFP (SEQ ID NO: 6, where X¹ is F and X² is F), GEGFP (SEQ ID NO: 6, where X¹ is E and X² is F) and GKGFP (SEQ ID NO: 6, where X¹ is K and X² is F), for example. Particularly preferred bioelastic materials are those that contain at least one GVGIP (SEQ ID NO: 6, where X¹ is V and X² is I) monomer, as the presence of at least one of these monomers has been shown to provide for a tougher matrix and one with a higher degree of hydrophobicity than GVGVP.

Examples of bioelastomers comprising one or more of these preferred monomer units include Polymers I though XV and Polymers I′ through XVI′, set forth herein, which list is intended to be exemplary and not limiting in any manner. Bioelastomers having increased hydrophobicity are best illustrated with Polymer III, Polymer VIII, Polymer IX, and Polymer XIII′ and XV′. The stepwise replacement of Val by Phe residues in GVGVP-based bioelastomers provides for the GVGFP, GEGFP and GFGFP pentamers of Polymer III and the GFGEP, GEGFP, GFGFP and GFGIP pentamers of Polymer VIII. Similarly, the stepwise replacement of Val by Tyr residues in GVGIP-based bioelastomers provides for the GYGIP pentamers of Polymers VII′ through XVI′ (SEQ ID NOS: 49 through 58).

As noted above, preferred bioelastic materials are comprised of “a-(GVGVP)ₙ-b” and of “a-(GVGIP)ₙ-b” monomer units, where n, a and b are as defined above. The Gibbs free energy in aqueous solutions of these (GVGVP)ₙ and (GVGIP)ₙ based polymers, is dependent on the proximity to charged moieties to hydrophobic moieties and arises due to a competition for hydration between charged (polar) and hydrophobic (apolar) moieties. The experimental basis for this conclusion derives from a series of experimental observations. First, by using microwave dielectric relaxation observation of aqueous solutions of properly designed bioelastomers, it was found that ionization of a carboxyl moiety dramatically decreases the amount of water of hydrophobic hydration. Second, stretching of hydrophobically folded bioelastomers that contain a limited number of carboxyl groups increases the pKa of carboxyl moieties. Third, the stepwise replacement of Val residues by more hydrophobic Phe residues in bioelastomers results in hydrophobic-induced increases in the pKa of carbonyl moieties. Fourth, an increase in the number of carboxylate moieties from zero to two, for every 100 residues, results in a decrease in the endothermic heat of the hydrophobic folding transition. Fifth, this same increase in the number of carboxylate moieties results in a dramatic increase in the temperature at which the hydrophobic folding transition occurs. Sixth, the temperature of the hydrophobic folding transition decreases as the amount of water of hydrophobic hydration increases.

### Hydrophobicity and Dissociation Rate

There are three issues to be addressed in designing bioelastomers that will be hydrophobically tunable low frequency absorbers by the ion pair dissociation mechanism. The increase in hydrophobicity must result in a sufficient decrease in the dissociation rate of the ion pair so as to decrease the frequency for surmounting the first barrier for dissociation. The stretching of ion-pair containing, hydrophobically folded and cross-linked bioelastomers decreases the frequency for jump over the initial dissociation barrier. Lastly, the jump over the proximal barrier for the ion-pair dissociation, which is observable as dielectric relaxation, must have a significant acoustic absorption counterpart. This last requirement is met, based upon the knowledge that the dissociation of the MgSO₄ divalent ion pair and salts of boronates are responsible for the significant low frequency acoustic absorption of seawater. See, Robert J. Urick, Principles Of Underwater Sound (Peninsula Publishers, 1996, ISBN 093146627). Another interesting observation is that ion-pairing of a positive cation with a negative carboxylate lowers the temperature of the hydrophobic folding and assembly transition, increases the amount of water of hydrophobic hydration, and lowers the Gibbs free energy for the system.

The competition for hydration between charged and hydrophobic moieties in protein-based polymers is a cooperative process, it has been found that increasing hydrophobicity by a stepwise replacement of Val by Phe residues in GVGVP monomers, increases ion-pair affinity as directly measured in terms of an increased binding constant and as observed in terms of decreased rates of ion-pair dissociation and increases in the steepness of the titration curve. The increase in ion-pair affinity causes a decrease in the rate of ion-pair dissociation. In addition, as the ion-pair distance increases, the complex becomes more polar.

Accordingly, it is expected that introducing an anionic or cationic site into a polymer, preferably an appropriately hydrophobic bioelastomer, will have the effect of increasing the bioelastomer's low frequency acoustic absorption, once a cation (or anion) binds the anionic (or cationic) site such that it is readily introduced into a bioelastomer by methods described herein, so that an ion-pair dipole is formed. Exemplary anion-cation pairs are polymer —COO⁻ . . . Na⁺, polymer-OSO₃⁼ . . . Mg⁺², and polymer-OPO₃⁼² . . . Mg⁺².

For protein based polymers such as elastomers, the ionization of the bioelastomer for introduction of an anionic site can readily be accomplished by ionization of a —COOH, carboxyl, moiety, i.e., using the carboxylate as a functional chain. The method of introducing an anionic site on a protein-based polymer such as a bioelastomer can involve sulfation of tyrosine-containing polymers. For example, a solution of polymer in trifluoroacetic acid (10 mL/gram) is treated with chlorosulphonic acid (1.5 equiv.) and left the reaction mixture for 30 min at room temperature. Adding ethanol destroys the excess reagent. The solvent is removed in vacuum. The residue is dissolved in H₂O, dialyzed and lyophilized. The method of introducing an anionic site on a protein-based polymer such as a bioelastomer can also occur by phosphorylation of a kinase site. For example, the kinase site RGYSLG can be included as part of the sequence of a protein-based polymer, and a cardiac cyclic AMP dependent protein kinase can be used to phosphorylate the serine (Ser, S) residue. This has been demonstrated in Pattanaik, et al., Biochem. Biophys. Res. Comm. 178:539-545 (1991). Also sulfation could be achieved enzymatically using the correct enzyme and peptide sequence incorporated into the protein-based polymer sequence.

Although ionization of a terminal carboxyl or amino moiety is possible in a bioelastomer comprised solely of GVGVP (SEQ ID NO: 6, where X¹ is V and X² is V) units, one is somewhat limited in the number of such sites and the ion pair selection, MgSO₄ being one of the limited candidates. Accordingly, it is preferred to include at least one GVGVP or at least one GVGIP analogue containing at least one residue that readily undergoes sulfonation, such as the aromatic containing tyrosine residue. In this manner, it is useful to include at least one GX¹GX²P (SEQ ID NO: 6) or GX⁴GX⁵P (SEQ ID NO: 24) monomer in the bioelastomer where X¹ and X² are as defined above; X⁴ is selected from the group consisting of V, E, F, Y, K, S (serine) and T (threonine); and X⁵ is selected from the group consisting of V, E, F, I, S, T and Y; with the proviso that at least one of X⁴ and X⁵ is Y, S, or T, such as in SEQ ID NOS: 41 and 49-58.

The invention also contemplates insertion of one or more, preferably one, serine (S), threonine (T) or tyrosine (Y) residues in the bioelastomer sequence. The insertion can be an addition between two monomers, for example, by means of illustration and not limitation, -GVGVP-X³-GVGVP- (SEQ ID NO: 23) where X³ is S, T or Y, or it can be positioned within a monomer unit such as described above for GX⁴GX⁵P (SEQ ID NO: 24). The hydroxyl group on serine and threonine, and the phenol on tyrosine are readily nitrated, sulfated or phosphorylated.

In general, the method of introducing an anionic site on a non-protein based polymer could involve simply poly(methylmethacrylate) with an incomplete methylation leaving free carboxylate functional groups, or poly(N-isopropyl acrylamide) with incomplete amide formation again leaving free carboxylate functional groups. The carboxylic functional groups can be sites for addition by amide linkage of an amino phenyl boronate and other aromatic groups with the capacity of adding anionic sites such as sulfates, phosphates, nitrates, etc.

Accordingly, in one aspect, the present invention provides for methods of making a polymer, preferably a bioelastomer, suitable for use as a low frequency acoustic absorber by modifying the polymer to include a charged (anionic or cationic) site, by which an ion-pair dipole is subsequently formed.

The cation in such ion-pair dipole is selected from the group consisting of alkali, alkaline earth, transition metal ions, lanthamides, actinides, and so forth, for example by way of illustration and not limitation, Na⁺, Ca⁺, Mg⁺², Ba⁺², Sr⁺² and Pb⁺², etc. The anion in such ion-pair dipole is selected from the group consisting of carboxylates, phosphates, sulfates, borates, silicates, and so forth, for example by way of illustration and not limitation, —COO⁻, —OSO₃⁻², —OPO₃⁻², —OBO₂⁻², and so forth.

Additionally the polymer could contain the cationic site as in the —NH₃⁺ of lysine (Lys, K) or the guanidinium of arginine (Arg, R), and the soluble ion would be the anion, such as, halides (Group VII elements) and oxidized Group VI elements, e.g., sulfates, and as phosphates, silicates, etc.

In yet another aspect of the invention, the bioelastomer could bind to a therapeutic agent such as a drug, vaccine, protein, antibody and so forth, for example, by ion-pairing, and sound absorption by the bioelastomer composition could be used to control the release of the therapeutic agent. In this manner, the means of controlling agent release is actually a form of free energy transduction where the input energy is sound and the output is in the form of chemical energy.

Aqueous solutions of bioelastomers based on the GVGVP (SEQ ID NO: 6, where X¹ is V and X² is V) pentapeptide and its analogues exhibit inherently interesting absorptions in the 1 MHz to 10 GHz range. In the range between 1 and 100 MHz, there exists an intense dielectric relaxation, Δε′≈70, due to rocking motions of the dipolar peptide moiety, and a relaxation of variable intensity near 5 to 10 GHz, which results from the reorientation of waters of hydrophobic hydration. These tunable relaxations become of relevance to absorption in radar frequencies.

Of most direct relevance to acoustic absorption are Polymers II (SEQ ID NO: 6, where X¹ is V and X² is I and n=260) and III (SEQ ID NO: 8). As shown in FIGS. 2 and 3, respectively, these bioelastomers exhibit intense acoustic absorptions that directly overlay the preferred sonar range of 200 Hz to 7000 Hz with absorptions that qualitatively and qualitatively are much preferred over representatives of the currently used polymers such as natural rubber and the polyurethane (PAN 10) data also included.

Accordingly, an object of the invention is the design and preparation of polymers that are capable of low frequency acoustic absorption in the range of up to 100 kHz. This is accomplished by first designing a polymer having the desired hydrophobicity, such as by selecting residues with enhanced hydrophobicity, and then introducing a charged site. Therefore, in one embodiment of the invention, a method of designing polymers that are capable of low frequency acoustic absorption, comprises the steps of: (a) forming a polymer comprised of repeating peptide monomeric units selected from the group consisting of nonapeptide, pentapeptide and tetrapeptide monomeric units, wherein said monomeric units form a series of β-turns separated by dynamic bridging segments suspended between said β-turns; and (b) further enhancing the acoustic absorption by introducing a charged site, such as an anionic or cationic site, on said polymer by the methods described herein. The polymer preferably comprises at least one pentapeptide monomeric unit having the formula GX¹GX²P (SEQ ID NO: 6), where X¹ is selected from the group consisting of V, E, F, Y and K; and X² is selected from the group consisting of V, E, F and I.

### Entropic Elasticity

By means of AFM (atomic force microscopy), studies by several research groups have been conducted and single chain stress/strain curves were obtained on titin (connectin), a single 3 million Da protein chain. Since this protein is mostly a series of 90-100 amino acid residue repeating sequences for most of the protein as well as 22-26 residue repeating sequences for more than a 2000 residue of the sequence, titin is a protein-based polymer composed of repeating peptide sequences. Accordingly, its traits presented a useful basis for the study of the bioelastomers and their usefulness in the methods of the invention

In the AFM experiment, one end of the single chain was attached to the cantilever tip and the other end to a surface. Force was measured as a function of chain length, and forces of a few pN/chain were determined. A single 3 million molecular weight protein chain, greater than 1000 nm in length and 4 nm in width, was demonstrated to be an entropic elastomer. Since the classical theory of rubber elasticity requires random chain networks, this work establishes that entropic elasticity can occur without random chain networks and without a random, or Gaussian, distribution of end to end chain lengths.

Contrary to the popular scientific dogma of the last half century as concerns the source of entropic elasticity being solely due to random chain networks, elastic protein-based polymers such as titin and the bioelastomers of the present invention can be regular dynamic structures and still be dominantly entropic elastomers. In fact, the differences between the bioelastomers and the rubbers of FIGS. 2 and 3, constitute a classic delineation between the two types of entropic elastomers and demonstrate the great utility of bioelastomers for design of selective acoustic absorption.

The decrease in entropy on extension provides for the entropic elasticity of elastic protein-based polymers of regular structure. This appears to result from one or both of two proposed mechanisms. One proposed mechanism is that a decrease in solvent entropy occurs upon extension. This occurs on the stretch-induced unfolding of hydrophobically folded protein-based polymers. The result of exposure of hydrophobic groups is formation of lower entropy water of hydrophobic hydration. The second proposed mechanism for entropic elasticity is based upon the damping of internal chain dynamics on extension, where large amplitude rocking motions of peptide moieties in the relaxed state become damped on extension. See Chang, et al., J. Computational Chem. 10:850-855 (1989); and Urry, “Thermally Driven Self-assembly, Molecular Structuring and Entropic Mechanisms in Elastomeric Polypeptides,” in Mol. Conformation and Biol. Interactions, Balaram and Ramaseshan, Eds., Indian Acad. of Sci., Bangalore, India, pp. 555-583 (1991). Large amplitude torsional oscillations of peptide moieties in a chain polymer define a large volume in configuration space, or when considering coordinate and conjugate momentum, define a large volume in N-dimensional phase space. This volume decreases dramatically on damping of internal chain dynamics resulting from extension. From statistical mechanics, entropy is directly proportional to volume in phase space, which can be expressed to include the product of the magnitude of the torsion angle oscillations. The decrease in amplitude of torsional oscillations, therefore, represents a large decrease in entropy for the protein-based polymer, represented in the instant invention as a repeating pentamer with fifteen backbone torsion angles in each pentamer, but with nine single-bond backbone torsion angles for which large torsion angle rotations are possible.

Accordingly, the instant invention is based upon the mechanism for entropic elasticity developed on poly(GVGVP)-type elastomers (SEQ ID NO:6, where X¹ is V and X² is V). This is referred to herein as the “librational entropy” mechanism of elasticity where the large amplitudes of peptide rocking motions of the unstretched state decrease as the chain is extended. The peptide moiety is the only source of dipole moment in poly(GVGVP) (SEQ ID NO: 6, where X¹ is V and X2 is V) and in poly(GVGIP) (SEQ ID NO: 6, where X¹ is V and X² is I). This, in combination with the otherwise developed molecular structure of poly(GVGVP) and the related poly(GVGIP), suggests the presence of large amplitude peptide rocking motions in the relaxed state. See Buchet, et al., J. Phys. Chem. 92: 511-517 (1988). Accordingly, dielectric relaxation becomes one experimental approach with which to examine this structural feature.

As described above in relation to FIGS. 1A-1D, the molecular structure of bioelastomers, for example the poly(GVGVP)-type (SEQ ID NO: 6, where X¹ is V and X² is V) protein-based elastomers, is one of a hydrophobically assembled helical array of β-turns connected by dynamic suspended segments. Referred to as a “β-spiral”, this conformation is an open helix with water inside. The suspended segments, which connect the β-turns, contain peptide moieties, which exhibit large amplitude rocking (“librational”) motions that become damped on extension. Thus, the suspended segments that link the β-turns function as entropic molecular springs. The β-turns are arranged on the surface of the helix with their planes parallel to the helix axis. In this structural configuration, the β-turns provide dynamically active surface elements, effectively forming a series of vibrating plates on the surface of the wide, water-containing and dynamic helix. A classical analogy would be the helical array of plates connected by springs. The β-turn surface elements can be thought of as molecular acoustic vanes attached to springs that are tunable to resonate over desired frequency ranges. The molecular structure and a graphical depiction illustrating the acoustic functional features are shown in FIGS. 1A-1D.

Numerous polymers were synthesized and evaluated:

Polymer I: (GVGVP)₂₅₁ (SEQ ID NO:6, where X¹ is V and X² is V and n = 251).

Polymer II: (GVGIP)₂₆₀ (SEQ ID NO:6, where C¹ is V and X² is I and n = 260).

Polymer III: (GVGVP GVGFP GEGFP GVGVP GVGFP GFGFP)ₙ (GVGVP), where n is 32 (SEQ ID NO:8).

Polymer IV: (GVGVP GVGFP GEGFP GVGVP GVGFP GVGFP)ₙ (GVGVP) where n is 41 (SEQ ID NO:9).

Polymer V: (GVGVP GVGVP GEGVP GVGVP GVGFP GFGFP)ₙ (GVGVP), where n is 39 (SEQ ID NO:10).

Polymer VI: (GVGVP GVGFP GEGFP GVGVP GVGVP GVGVP)ₙ (GVGVP), where n is 40 (SEQ ID NO:11).

Polymer VII: (GVGVP GVGVP GEGVP GVGVP GVGVP GVGVP)ₙ (GVGVP), where n is 36 (SEQ ID NO:12).

Polymer VIII: (GVGIP GFGEP GEGFP GVGVP GFGFP GFGIP GVGIP GFGEP GEGFP GVGVP GFGFP GFGIP)ₙ (GVGVP), where n is 20 (SEQ ID NO:13).

Polymer IX: (GVGVP GVGFP GKGFP GVGVP GVGFP GFGFP)ₙ (GVGVP), where n is 21 (SEQ ID NO:14).

Polymer X: (GVGVP GVGFP GKGFP GVGVP GVGFP GVGFP)ₙ (GVGVP), where n is 21 (SEQ ID NO:15).

Polymer XI: (GVGVP GVGVP GKGVP GVGVP GVGFP GFGFP)ₙ (GVGVP), where n is 22 (SEQ ID NO:16).

Polymer XII: (GVGVP GVGFP GKGFP GVGVP GVGVP GVGVP)ₙ (GVGVP), where n is 25 (SEQ ID NO:17).

Polymer XIII: (GVGVP GVGVP GKGVP GVGVP GVGVP GVGVP)ₙ (GVGVP), where n is 35 (SEQ ID NO:18).

Polymer XIV: (GVGVP GVGFP GEGFP GVGVP GVGFP GKGVP)ₙ (GVGVP), where n is 21 (SEQ ID NO:19).

Polymer XV: (GVGVP GVGFP GEGFP GVGVP GVGVP GKGVP)ₙ (GVGVP), where n is 21 (SEQ ID NO:20).

Although the values of the integer “n” are provided above, it is understood that that is illustrative of the polymers of the invention and is not intended to be limiting. Polymers having repeating units such as those described for Polymers I-XV can be designed to have n values within the range of 1 to 5000. The latter value is reflected in the size of titin, the largest protein (3,000,000 Da) known to be biologically synthesized.

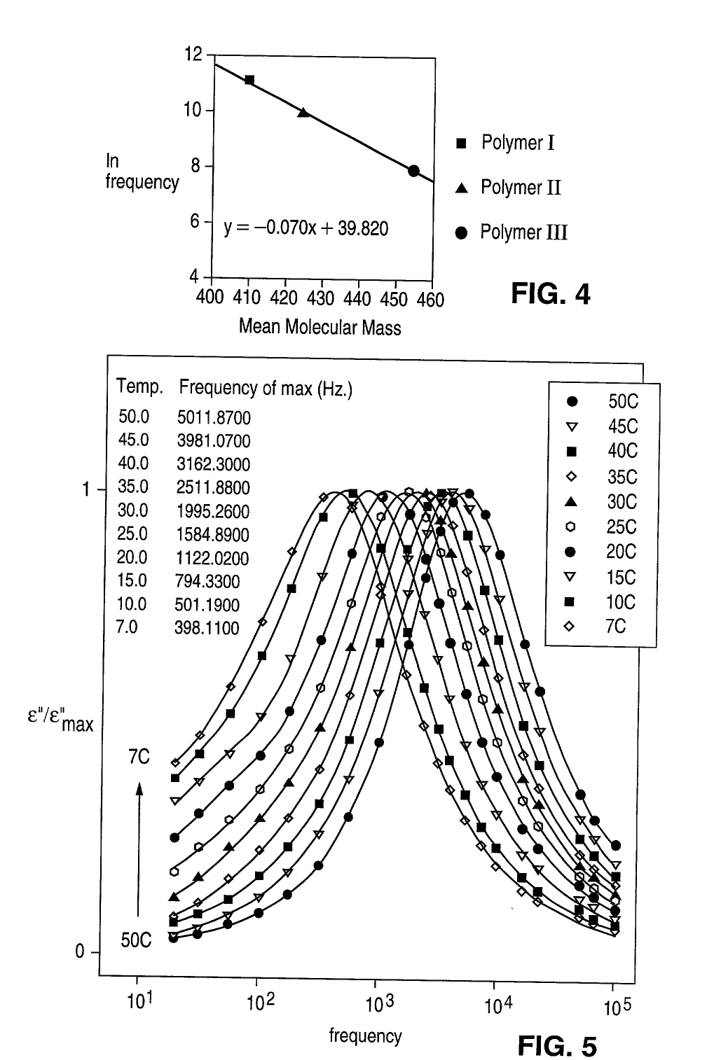

Low frequency acoustic dielectric relaxation data observed for Polymers I, II and III of FIG. 4, have demonstrated the capacity to design and build a broad spectrum of acoustic and dielectric absorptions in the range of 100 Hz to 100 kHz. In particular, it has been found that polymers suitable for use as low frequency acoustic absorbers in the methods of the invention exhibit at least one, and preferably more of the following characteristics, which are described in detail herein:

(a) A mean mass of the repeating unit within the range of 300 to 10,000 daltons (Da).

(b) A cross-link density of the matrix within the range of one cross-link per 300 dalton, to one per 40,000 dalton of polymer and even to no cross-links between chains (coacervate state).

(c) A water content of the matrix or coacervate within the range of 2% to 99% by weight.

(d) A hydrophobicity of the repeating unit within the Tₜ range of −200° C. to 90° C., preferably within the Tₜ range of −100° C. to 90° C.

(e) A transition temperature within the range of −200° C. to 120° C., preferably within the range of −100° C. to 120° C.

The mean mass of the repeating unit is calculated by for Polymers III through XV by summing the molecular weight encoded by the basic monomer gene and then dividing by the number of pentamers (6) in the 30 mer peptide repeat encoded by the basic monomer gene.

The cross-link density of the matrix is determined by measuring the extent of swelling on lowering the temperature, and the data below Tₜ is analyzed using random chain network theory. Alternatively, the change in shear modulus on swelling is determined and similarly analyzed. Analytically, when cross-linking by means of chemical coupling of carboxyl side chains with amino side chains involving Polymers III through XIII and using the water soluble carbodiimide approach as outlined above, the analytical determination of remaining free carboxyl groups and free amino groups becomes possible by acid-base titration data. This experimental data can then be used to check the swelling theoretical approaches just noted.

The water content of the matrix or coacervate is determined and measured as previously published (Urry, et al., Biopolymers 24:2345-2356 (1985)) by a combination of volumetric, lyophilization and weighing approaches.

The hydrophobicity of the repeating unit is determined by amino acid selection, as described herein. The transition temperature is determined by the method described above for determining Tₜ and by differential scanning calorimetry (DSC) to measure the onset of the endothermic inverse temperature transition. The enthalpy and entropy values so determined by DSC provide another measure of hydrophobicity. See Urry, “Physical Chemistry of Biological Free Energy Transduction as Demonstrated by Elastic Protein-based Polymers,” invited FEATURE ARTICLE, J. Phys. Chem. B 101:11007-11028 (1997). Yet another measure of hydrophobicity is the use of microwave dielectric relaxation to determine the amount of hydrophobic hydration. See Urry, et al., JACS 119:1161-1162 (1997).

Values for some of the bioelastomers of the invention are set forth in the table below:

| | Mean mass | Cross-link | Water | Hydrophob- | Transition |
|---|---|---|---|---|---|
| | of the | density of | content of | icity of the | temperature, |
| | repeating | the matrix | the | repeating | experimental, |
| Polymer | unit (Da) | (max)¹ | coacervate² | unit³ | water⁴ |
| I | 409.5 | 1:2000 Da | 60% by wt | Tₜ = 25° C. | Tₜ of 27° C. |
| II | 423.5 | 1:2000 Da | 45% by wt | Tₜ = 10° C. | Tₜ of 12° C. |
| III | 454.5 | 1:5000 Da | <30% by wt | Tₜ <0° C. | Tₜ of 55° C. |
| IV | 446.5 | 1:5000 Da | ˜35% by wt | Tₜ = 8.5° C. | Tₜ >80° C. |
| V | 438.5 | 1:5000 Da | ˜40% by wt | Tₜ = 13° C. | Tₜ >80° C. |
| VI | 430.5 | 1:5000 Da | ˜45% by wt. | Tₜ = ˜40° C. | Tₜ >80° C. |
| VII | 414.5 | 1:5000 Da | 60% by wt | Tₜ = 59° C. | Tₜ >80° C. |
| VIII | 464.2 | 1:5000 Da | <30% by wt | pH & salt | Tₜ >95° C. |
| | | | | dep. | |
| IX | 454.4 | 1:5000 Da | ˜30% by wt | Tₜ <0° C. | Tₜ of 15° C. |
| X | 446.4 | 1:5000 Da | ˜35% by wt | Tₜ <0° C. | Tₜ of 24° C. |
| XI | 438.4 | 1:5000 Da | ˜40% by wt | Tₜ = 5° C. | Tₜ of 33° C. |
| XII | 430.4 | 1:5000 Da | ˜45% by wt | Tₜ = 17° C. | Tₜ of 57° C. |
| XIII | 414.3 | 1:5000 Da | ˜60% by wt | Tₜ = 37° C. | Tₜ >80° C. |
| XIV | 443.3 | 1:2500 Da | ˜40% by wt | pH and salt | Tₜ of 17° C. |
| | | | | dependent | |
| XV | 435.3 | 1:2500 Da | ˜45% by wt | pH and salt | Tₜ of 29° C. |
| | | | | dependent | |
| ¹A maximal cross-link density given in terms of cross-link to molecular weight of chain. | | | | | |
| ²The water content is given as that of the coacervate state; it is also that of the contracted (de-swollen) state of a cross-linked matrix. The swollen state of a cross-linked matrix depends on the chain length and the degree of cross-linking and can be as high as 99% by weight. Values of 90% are routinely obtained for 20 Mrad cross-linked chains of 50,000 to 100,000 Da. | | | | | |
| ³These are the values revelant to the hydrophobicity scale in which the conditions are for phosphate buffered saline (0.15 N NaCl, 0.01 M phosphate, pH 7.5). The Tₜ values are highly pH and salt dependent. Adding salt lowers Tₜ, as does protonation of a carboxylate or deprotonation of an ammonium. In general, without salts at acid pH, e.g., pH of 3, the Tₜ values of E-containing polymers will be lower and often well below 0° C. with 3 or more F residues per 30 mer, | | | | | |
| # whereas at neutral pH (7.5) the value of Tₜ will be greater than 80° C. with 3 or fewer F residues per 30 mer. The inverse shifts occur for K-containing polymers but with smaller changes in Tₜ as a function of pH. | | | | | |

⁴The values are given for neutral pH (7.5) in water.

The compositions of the primed series are, where “n” can be an integer from about 1 to 5000:

| | | |
|---|---|---|
| Polymer I′ | [(GVGIP GEGIP GVGIP)₃]ₙ | SEQ ID NO:29 |
| Polymer II′ | [(GVGIP GVGIP GEGIP GVGIP GVGIP GVGIP)]ₙ | SEQ ID NO:30 |
| Polymer III′ | [(GEGIP GVGIP GEGIP GVGIP GVGIP GVGIP)]ₙ | SEQ ID NO:31 |
| Polymer IV′ | [(GVGIP GKGIP GVGIP)₃]ₙ | SEQ ID NO:32 |
| Polymer V′ | [(GVGIP GVGIP GKGIP GVGIP GVGIP GVGIP)]ₙ | SEQ ID NO:33 |
| Polymer VI′ | [(GKGIP GVGIP GKGIP GVGIP GVGIP GVGIP)]ₙ | SEQ ID NO:34 |
| Polymer VII′ | [(GVGIP)₂₁(GYGIP)]ₙ | SEQ ID NO:35 |
| Polymer VIII′ | [(GVGIP)₂₁(GY{SO₄⁼}GIP)]ₙ | SEQ ID NO:36 |
| Polymer IX′ | [(GVGIP)₁₁(GYGIP)]ₙ | SEQ ID NO:37 |
| Polymer X′ | [(GVGIP)₁₁(GY{SO₄⁼}GIP)]ₙ | SEQ ID NO:38 |
| Polymer XI′ | [(GVGIP)₈(GYGIP)]ₙ | SEQ ID NO:39 |
| Polymer XII′ | [(GVGIP)₈(GY{SO₄⁼}GIP)]ₙ | SEQ ID NO:40 |
| Polymer XIII′ | [(GVGIP)₅(GYGIP)]ₙ | SEQ ID NO:41 |
| Polymer XIV′ | [(GVGIP)₅(GY{SO₄⁼}GIP)]ₙ | SEQ ID NO:42 |
| Polymer XV′ | [(GVGIP)₂(GYGIP)]ₙ | SEQ ID NO:43 |
| Polymer XVI′ | [(GVGIP)₂(GY{SO₄⁼}GIP)]ₙ | SEQ ID NO:44 |

| | Mean mass | Cross-link | Water | Hydrophob- | |
|---|---|---|---|---|---|
| | of the | density of | content of | icity of the | |
| | repeating | the matrix | the | repeating | Transition |
| Polymer | unit (Da) | (max)¹ | coacervate² | unit³ | temperature |
| I′ | 433.5 | 1:2500 Da | ˜45% by wt | pH dep. | pH dep. |
| II′ | 428.5 | 1:5000 Da | ˜45% by wt | pH dep. | pH dep. |
| III′ | 433.5 | 1:2500 Da | ˜45% by wt | pH dep. | pH dep. |
| IV′ | 433.1 | 1:2500 Da | ˜45% by wt | pH dep. | pH dep. |
| V′ | 428.3 | 1:5000 Da | ˜45% by wt | pH dep. | pH dep. |
| VI′ | 433.1 | 1:2500 Da | ˜45% by wt | pH dep. | pH dep. |
| VII′ | 426.4 | 1:2000 Da | ˜45% by wt | pH dep. | pH dep. |
| VIII′ | 430.1 | 1:2000 Da | ˜50% by wt | pH dep. | pH dep. |
| IX′ | 428.8 | 1:2000 Da | ˜45% by wt | pH dep. | pH dep. |
| X′ | 435.5 | 1:2000 Da | ˜50% by wt | pH dep. | pH dep. |
| XI′ | 430.6 | 1:2000 Da | ˜40% by wt | pH dep. | pH dep. |
| XII′ | 439.5 | 1:2000 Da | ˜50% by wt | pH dep. | pH dep. |
| XIII′ | 434.2 | 1:2000 Da | ˜40% by wt | pH dep. | pH dep. |
| XIV′ | 447.5 | 1:2000 Da | ˜55% by wt | pH dep. | pH dep. |
| XV′ | 444.9 | 1:2000 Da | ˜35% by wt | pH dep. | pH dep. |
| XVI′ | 471.5 | 1:2000 Da | ˜60% by wt | pH dep. | pH dep. |
| ¹A maximal cross-link density in terms of cross-link to molecular weight of chain. | | | | | |
| ²The water content is given as that of the coacervate state; it is also that of the contracted (de-swollen) state of a cross-linked matrix. The swollen state of a cross-linked matrix depends on the chain length and the degree of cross-linking and can be as high as 99% by weight. Values of 90% are routinely obtained for 20 Mrad cross-linked chains of 50,000 to 100,000 Da. | | | | | |
| ³Indicates highly pH and salt dependent. The values are given for neutral pH (7.5) in water. Adding salt lowers Tₜ as does protonation of a carboxylate or deprotonation of an ammonium. In general, at acid pH, e.g., pH of 3, the Tₜ values of E-containing polymers will be lower and often with below 0° C. with 3 or more F residues per 30 mer, whereas at neutral pH (7.5) the value of Tₜ will be greater than 100° C. with 3 or fewer F residues per 30 mer. The inverse | | | | | |
| # shifts occur for K-containing polymers but with smaller changes in Tₜ as a function of pH. | | | | | |

Accordingly, one embodiment of the invention pertains to a method of designing polymers that are capable of low frequency acoustic absorption, which comprises forming a polymer comprised of repeating peptide monomeric units selected from the group consisting of nonapeptide, pentapeptide and tetrapeptide monomeric units, wherein said monomeric units form a series of β-turns separated by dynamic bridging segments suspended between said β-turns, wherein said polymer has one or more of the following characteristics: a mean mass of the repeating unit within the range of 300 to 10,000 daltons; a cross-link density of the matrix within the range of one cross-link per 300 dalton to no cross-links between chains (coacervate state); a water content of the matrix or coacervate within the range of 2% to 99% by weight; a hydrophobicity of the repeating unit within the Tₜ range of −200° C. to 90° C.; and a transition temperature within the range of −200° C. to 90° C. Another embodiment of the invention pertains to the polymers themselves.

### Synthesis

In order to obtain high molecular weight polymers in good yields, a number of approaches are available. Synthesis of the bioelastic repeating units is straightforward and easily accomplished by a peptide chemist or by standard methods in recombinant DNA technology and microbial fermentation. For example, organic synthesis of the polymers has been described in the patents incorporated herein by reference. In particular, the synthesis and cross-linking of poly(GVGVP) have been described in U.S. Pat. No. 4,783,523. The synthesis of poly(IPGG) has been described in U.S. Pat. No. 5,250,516 and that of poly(GGAP) in U.S. Pat. No. 5,527,610. Accordingly, the teachings of these patents can be applied to the synthesis of bioelastic polymers having different monomer units. When producing polymers by chemical synthesis, care should be taken to avoid impurities, because small levels of impurities can result in termination of the polymerization process or in racemerization that can alter the physical properties of the resulting polymer, but there are otherwise no particular problems of synthesis. Peptide unit purity is important in obtaining a material with suitable physical properties since, for example, small changes in the preparation of the bioelastomers can result in a Tₜ that varies as much as 15° C. The solution of this potential problem is simply to purify the components used to prepare the peptide.

The polymer can be prepared as a homopolymer or a copolymer. Either random or block copolymers prepared from at least two of the monomeric units are useful in the methods of the present invention but are less preferred when an equivalent homopolymer has the desired physical properties, simply because of the greater complexity of synthesis. Irrespective of how the bioelastic polymers are synthesized, these can further be derivatized, if desired. For example, electroresponsive side chains can be incorporated into the polymer as described in Urry, U.S. Pat. No. 5,900,405 (electrical exposure).

The protein-based polymers can also be prepared using genetic engineering techniques. Using this approach, a gene encoding the desired peptide sequence is constructed, artificially inserted into, and then translated in a host organism. The organism can be prokaryotic, e.g., bacterial, or eukaryotic, e.g., yeast or plants. Techniques are known in the art of molecular biology to manipulate genetic information (i.e., DNA sequences) for effective gene expression in an appropriate host organism (see, for example, Sambrook et al., Molecular Cloning: A Laboratory Manual, Second Edition, Cold Spring Harbor, N.Y. (1989)) and include the use of enzymes capable of cleaving, joining, copying or otherwise modifying polynucleotides. In addition, vectors allowing the introduction of this information into the host organism in a suitable manner for expression are known in the art. A detailed example of the production of poly-VPGVG is set out in McPherson, et al., Biotechnol. Prog. 8:347-352 (1992), and McPherson, et al., Protein Expression and Purification 7:51-57 (1996), publications arising from the laboratory of the present inventor. These publications can be used as guidance for genetic-based production of any of the materials used in the present invention. Polymers with as many as 2000 amino acid residues have been expressed in good quantity by appropriate E. coli strains. For example, expression of (GVGVP)₁₂₁ (SEQ ID NO: 6, where X¹ is V and X² is V and n=121) has occurred at levels of 80% of E. coli cell volume. Accordingly, many of the bioelastomers suited for use in the methods of the invention can be synthesized at low cost by use of E. coli, transformed to produced (GVGIP)₂₆₀ (SEQ ID NO: 6, where X¹ is V and X² is I and n=260), the expression vector which is then modified for co-expression of an easily measured expression product.

The gene constructions of Polymers IX′ and VII′ are given as examples of the primed series. The monomer gene encoding Polymer IX′, [(GVGIP)₁₁(GYGIP)]ₙ are constructed in three steps. Initially, the gene encoding for (GVGIP)₁₀ is constructed and its sequence verified. Then the sequence for (GVGIP) (GYGIP) is made and attached to (GVGIP)₁₀. Finally, the whole unit, (GVGIP)₁₁(GYGIP) is cloned and sequenced again to verify the authenticity of the entire coding sequence.

To construct (GVGIP)₁₀, two single-stranded oligonucleotides encoding the two halves of (GVGIP)₁₀ are chemically synthesized by a commercial source. The two opposing oligonucleotides then are annealed at their 3′ ends through a 20 base pair complimentary region, and extended to the full length, double stranded basic gene fragment by a high fidelity thermostable DNA polymerase. The resulting gene fragment is digested by the restriction enzyme BamHI before its insertion into the cloning vector pUC118 through ligation. After the transformation of the cloning host E coli DH5aF′, the positive clones are recovered from the selection plates and the plasmid DNA from each clone is isolated for the screening analysis. The resulting plasmids are digested with BamHI and separated on an agarose gel. The clones containing an insert of about 180 base pairs in length are the candidates for subsequent sequence verification. Once the sequence is verified, this clone is used as a source of (GVGIP)₁₀for the ensuing gene construction. Similarly the two oligonucleotides encoding (GVGIP) (GYGIP) are also chemically synthesized. Upon annealing, this double stranded, full length DNA fragment is ligated to (GVGIP)₁₀ and cloned into vector pUC118. The possible clones are recovered from selection plates and the sequences of the positive clones are analyzed for bearing the correct sequence encoding monomer gene (GVGIP)₁₀(GVGIP) (GYGIP). This clone is then used as the source of monomer gene fragment for construction of concatemer genes.

The monomer gene encoding polymer VII′ (GVGIP)₂₁(GYGIP) is constructed in the similar three-step fashion. The resulting monomer gene has the sequence encoding (GVGIP)₁₁(GYGIP) (GVGIP)₁₀.

The construction of monomer genes encoding polymer XI′, XIII′, and XV′ (GVGIP)₂₁(GYGIP) are the same as for (GVGIP)₁₀. The resulting monomer genes have the sequences encoding (GVGIP)₈(GYGIP), (GVGIP)₅(GYGIP), and [(GVGIP)₂(GYGIP)]₃, respectively.

To construct the concatemer (multimer) gene, a large amount of the monomer gene fragments are prepared after digesting the monomer gene containing plasmid with the restriction enzyme. The resulting monomer gene fragments are concatenated (ligated) in the presence of N- and C-terminal adaptors, which provide the cloning sites for the subsequent manipulation in different vectors. The resulting concatenation products, consisting of multimer genes of varying chain length, are ligated into pUC118 and introduced into E Coli. The possible clones on the selection plates are screened and the positive clones identified after a series of digestion with different restriction enzymes.

By using recombinant DNA technology, the cost of production of bioelastomers can be competitive with synthetic, organic polymers and with natural materials that need extensive purification. Producers of industrial proteins have demonstrated that costs can be reduced significantly for biologically produced proteins. Cost is of particular importance when producing bioelastomers for use as acoustic absorbers, due to the large quantities needed.

The bioelastic polypeptide can be purified, for example, from cultures grown in fermentation reactors or from organic syntheses, by its ability to undergo an inverse temperature transition. Purification using the inverse temperature transitional properties of the protein-based polymers is preferred with genetically engineered polymers expressed in microbial systems as even endotoxin levels have been demonstrated to be particularly reduced using this method. See Urry, et al., J. Biomater. Sci. Polymer Edn. 9:1015-1048 (1998).

### Methods of Use

One aspect of the invention pertains to a method for reducing the acoustical noise, reducing the sonar cross-section or reducing the radar cross-section of an object, comprising covering the object with a polymer which comprises repeating peptide monomeric units selected from the group consisting of nonapeptide, pentapeptide and tetrapeptide monomeric units, wherein said monomeric units form a series of β-turns separated by dynamic bridging segments suspended between said β-turns.

As used herein the term “reducing the acoustical noise” of an object is intended to mean that the ability of the object to absorb sound from an external source is increased, thereby reducing or eliminating the amount of sound waves that are reflected off the surface of the object or that can pass through an object. This is of particular utility when using the polymers of the invention to cover walls or as ear plugs, for example.

As used herein the term “reducing the sonar cross-section” of an object is intended to mean a reduction or elimination in the amount of sound waves that are reflected off a surface. This is of particular utility when using the polymers of the invention to coat submarines or the hulls of ships.

As used herein the term “reducing the radar cross-section” of an object is intended to mean a reduction or elimination in the amount of radiofrequency waves or microwaves that are reflected off a surface, i.e., an increase in the amount of such waves that are absorbed by the surface. This is of particular utility when using the polymers of the invention to coat submarines or the hulls of ships.

One embodiment of the invention, relates to a method for measuring the sound absorption capabilities of a protein-based material, and comprises the steps of: (a) forming a test component from a polymer comprising repeating peptide monomeric units selected from the group consisting of nonapeptide, pentapeptide and tetrapeptide monomeric units, wherein said monomeric units form a series of β-turns separated by dynamic bridging segments suspended between said β-turns, wherein the polymer is optionally modified to include a charged site; (b) exposing the test component to a high intensity, low frequency sound; (c) measuring the dielectric constant of the test component; and (d) correlating the measured dielectric constant of the test component to the level of sound that would be absorbed by a protein-based material when exposed to the high intensity, low frequency sound.

This method finds particular utility in evaluating the impact of sound absorption on protein-based materials, such as marine mammal and other biological tissues. There is much concern over the impact that high intensity, low frequency sounds (such as are produced by submarines) have on marine life, particularly mammals. State of the art methods of evaluating tissue damage involve rather crude experiments and the examination of damage to dead dolphin tissue from explosions or intense sound.

The acoustic absorbing bioelastomers described herein find particular utility as research tools in this area since they are ideal materials for the simulation of tissue from marine mammals such as dolphins and whales. These bioelastomers are similar to real animal tissue in texture, tensile strength and composition, along with having transduction properties that can be used as acoustical and pressure sensors. In addition, real time data is obtainable when using bioelastomers instead of dead animal tissue, with the further expectation of obtaining better data overall.

Accordingly, the invention also provides for a method of evaluating the impact of sound absorption on protein-based materials comprising the steps of: (a) forming a test component from a polymer comprising repeating peptide monomeric units selected from the group consisting of nonapeptide, pentapeptide and tetrapeptide monomeric units, wherein said monomeric units form a series of β-turns separated by dynamic bridging segments suspended between said β-turns; (b) exposing the test component to a high intensity, low frequency sound; (c) measuring the dielectric relaxation of the test component; and (d) correlating the measured dielectric relaxation of the test component to the damage that would occur to the protein-based materials when exposed to the high intensity, low frequency sound. The polymer can be optionally modified to include an ion-pair dissociation site with a hydrophobically tuned absorption of the desired frequency range. The polymer can sense the sound and provide a measure of the intensity in the output measured by dielectric activation.

In another embodiment of the invention, the polymers described herein can be used to absorb microwave and radar frequencies in furtherance of the development of stealth surface ships and in addition to measure microwave and radar exposure. There is much concern over the amount of microwave and radar exposure of persons working aboard ships and submarines, along with persons working at facilities that use microwave and radar energy. State of the art methods of measuring microwave and radar exposure utilize costly dose meters, whose detector diodes are generally only useful for frequencies well below 1 GHz.

The acoustic absorbing polymers described herein find particular utility as tools for the measurement of microwave and radar energy levels, i.e., as microwave dosimeters. These polymers, and in particular, the bioelastomers have a natural rf absorption capacity within the range of 1 MHz up to 20 GHz. As described above, the bioelastomers described herein undergo an inverse temperature transition. Accordingly, the heating effect from microwaves directed from a receiving antenna causes the bioelastomer to contract in direct proportion to the amount of microwave radiation received by the bioelastomer. The size or tension of the bioelastomer is then measured, which provides a reading of the rf environment. The preferred devices in such cases can be fibers of a range of diameters from nanometers to millimeters. Alternatively, the relaxation near 10 MHz can be monitored as an even more sensitive measure of microwave energy.

Accordingly, one embodiment of the invention pertains to a method of measuring the microwave or radar absorption capabilities of a material comprising the steps of: (a) forming a test component from a polymer comprising repeating peptide monomeric units selected from the group consisting of nonapeptide, pentapeptide and tetrapeptide monomeric units, wherein said monomeric units form a series of β-turns separated by dynamic bridging segments suspended between said β-turns, wherein the polymer is optionally modified to include a charged site; (b) exposing the test component to microwave or radar exposure; (c) measuring the dielectric relaxation of the test component; and (d) correlating the dielectric relaxation to the amount of microwave or radar absorption that would occur when the material is exposed to microwaves or radar. This method can be used to measure the radar absorption capabilities of a protein-based material or used to measure the microwave absorption capabilities of polymers with hydrophobic hydration.

The invention also pertains to methods for reducing noise pollution, whereby a person's ears are covered with an object (e.g., ear muffs) or an object is positioned within the interior of the ear (e.g., ear plugs), such object being made of a polymer which comprises repeating peptide monomeric units selected from the group consisting of nonapeptide, pentapeptide and tetrapeptide monomeric units, wherein said monomeric units form a series of β-turns separated by dynamic bridging segments suspended between said β-turns. Also encompassed by the invention are ear plugs, ear muffs and so forth, manufactured of the polymers described herein.

### Kits

The instant invention also contemplates packaging the polymers described herein as a kit for reducing acoustical noise either reaching or emanating from a site. Such a kit would contain a polymer of the invention, optionally modified to include an ion-pair dissociable site and a material to form a protective barrier. The kit may also contain a means for directly coating the object such as a brush or sprayer, a means for applying the protective barrier, a cross linking agent, a means for applying the cross lining agent, and any necessary instructions that would facilitate the use of the invention.

In a preferred embodiment, a kit for reducing the acoustical noise, reducing the sonar cross-section or reducing the radar cross-section of an object, comprises: (a) a polymer comprised of repeating peptide monomeric units selected from the group consisting of nonapeptide, pentapeptide and tetrapeptide monomeric units, wherein said monomeric units form a series of β-turns separated by dynamic bridging segments suspended between said β-turns, wherein said polymer has one or more of the following characteristics: a mean mass of the repeating unit within the range of 300 to 10,000 daltons; a cross-link density of the matrix within the range of one cross-link per 300 dalton to no cross-links between chains (coacervate state); a water content of the matrix or coacervate within the range of 2% to 99% by weight; a hydrophobicity of the repeating unit within the Tₜ range of −200° C. to 90° C.; and a transition temperature within the range of −200° C. to 90° C.; and (b) a material to form a protective coating. The kit of may further comprise a suitable cross-linking agent.

One material useful for forming the protective coating is a sheet of elastomer to prevent drying of the bioelastomer sheet, for example, a random chain elastomer such as polyurethane. The protective coating can protect the bioelastomer from sea water and may also serve to provide a non-reflecting surface. There may be a number of bioelastomer sheets and random chain elastomer sheets arranged in sandwich fashion.

### Methods of Application

The object whose acoustic characteristics are being modified can be contacted with the polymers of the invention by coating the surface with a viscous polymer composition, which is then allowed to cure to a tacky or gel state by drying at room temperature or at an elevated temperature, for example by exposure to a heat lamp. The typical thickness of a single layer of such a coating will be within the range of 0.01 to 12 inches. The coating can be accomplished by brushing or spraying the polymer composition onto the surface of the object. In some instances, it may be preferable to form the polymer in the configuration of a flexible sheet, that is positioned on the object's surface so as to conform to the shape of the surface, and then adhered in place. When formed in sheets, it is preferable to adhere the sheets to the hull so that the edges of the sheets overlap to some degree, to insure that there is no exposed hull surface. As noted above, the object can be a submarine and the surface can be the outer hull or the walls of one or more of the machinery rooms contained within the submarine.

It is important that the polymer not be allowed to completely dry, otherwise it will not function properly in the methods of the invention. By retaining the polymer in a tacky or gel state, there remains some level of water present, preferably within the range of 5-70 wt % water. It is this water content that allows motional modes and for continuous ion-pair dissociation/association to occur, and accordingly, continuous acoustic absorption. Water also allows for the absorption of the compressional waves by motion of the β-turn acoustic vane.

Since the polymer will not be hard, a protective coating is recommended to both maintain the water content of the polymer and to protect the tacky coating from chemical damage by the elements such as salt water and physical damage such as scratches from objects hitting the polymer coating. Since the purpose of the polymer is acoustic absorption, the protective coating must be either non-reflecting, i.e., transparent to frequency or it must absorb frequency. If the protective coating is reflective to any significant extent, it will limit to that extent frequencies from reaching the polymer coating, where they can be absorbed. More importantly, such frequencies will be reflected to their source, thus defeating the purpose of having an underlying acoustic absorbing coating. There are numerous materials that can be used as non-frequency reflecting protective coating, such as polypropylene and polyurethane.

In one embodiment of the invention, the polymer is positioned on the hull as a series of layers, each layer being optimized over a different part of the desired frequency range for absorption. Each layer is designed to cover the required frequency range to account for temperature and pressure changes, that is, there may be one composition layer for low temperature and high pressure and another for higher temperature and lower pressure. Each layer may be sandwiched between and sealed within protective rubber-like, e.g. polyurethane, sheets, with a total thickness of up to 12 inches. As noted above, the polyurethane acts as a sealant to maintain the water content of the polymer and as a protective coating to protect the polymer from chemical and physical damage.

In another embodiment of the invention, polymers having deep absorption dips at different frequencies are combined into a composite to optimize the desired absorption characteristics. In this manner, the material used in the method for reducing acoustical noise can be a single polymer or it may be a composite material comprised of two or more different polymers.

The following examples describe specific aspects of the invention to illustrate the invention and provide a description of the present method for those of skill in the art. The examples should not be construed as limiting the invention, as the examples merely provide specific methodology useful in understanding and practice of the invention.

### EXAMPLES

The preparation of the elastic protein-based polymers utilizes gene construction, development of an expression system, fermentation and purification as described for the particular examples given below. As for gene construction, the basic monomer genes were designed to have appropriate cohesive ends comprising an appropriate restriction site sequence (selected using standard techniques, such as consideration of the restriction sites present in the vector to be used for expression). Polymerization of the basic gene was carried out through the compatible sticky ends generated by restriction endonuclease digestion, followed by subsequent ligation using DNA ligase to form multimers of the basic gene. This protocol was used successfully to produce each of the monomer gene sequences. The monomers were then concatemerized (polymerized) to form multimer genes with many different numbers of repeats, and many of the multimer genes have been expressed at high levels as will be briefly reported below.

### Example 1

### Preparation of Materials

A. Phenylalanine and Lysine-Containing Pentameric Bioelastic Materials

This example illustrates a phenylalanine and lysine-containing bioelastic material. Generally, the protocols used for this particular construct is applicable to other protein elastomers described herein. Standard amino acid abbreviations are used here and elsewhere in this specification.

The polymer has the sequence [(GVGVP)₂ GKGVP GVGVP GVGFP GFGFP)]ₙ GVGVP, where n=22 (SEQ ID NO: 16), referred to herein as Polymer XI.

1. Gene Construction

The basic monomer gene for (GVGVP)₂ GKGVP GVGVP GVGFP GFGFP) (SEQ ID NO: 25) is constructed using the following oligonucleotides:

| | |
|---|---|
| 5′-GAGGATCCAGGCGTTGGGGTACCGGGTGTTGGC | (SEQ ID NO:26) |
| GATCCGGGTAAAGGTGTCCCGGGGTTGGTGTGC-3′ | |
| and | |
| 5′-CTGGATCCAACGCCTGGGAATCCGAAACCCGGA | (SEQ ID NO:27) |
| AAGCCTACACCCGGCACACC AACGCCCGGGACA-3′ | |

The oligos are annealed through the 20-base-pair complementary regions at their 3′ ends and extended with a thermostable DNA polymerase and free deoxy-nucleotides to give a double-stranded monomer gene, flanked on each end by both of the restriction endonuclease sites BamH1 and Bsp1. The basic monomer gene was cloned into pUC 118 via the BamH1 restriction site. After cloning and characterization of the monomer gene, it was digested out of pUC 118 with Bsp1 and purified in adequate amounts for polymerization. Polymerization was achieved by ligation using DNA ligase to join the gene fragments in a head-to-tail fashion through the non-palindromic Bsp1 ends and in the presence of cloning adaptors, thus forming multimers of the basic gene.

The gene sequences chosen allowed concatamerization of the basic gene sequence such that the gene segments remained in proper reading frame. Typical ligation conditions were used and the sequence of the basic concatemer gene was verified using routine procedures, such as with Sequenase™ from Amersham Life Science, Inc. Genes encoding bioelastic polymers as large as 2000 residues have been expressed using this technique.

2. Gene Expression and Fermentation

i. Expression

The pET plasmids (Novagen, Inc., Madison, Wis.)), such as pET-11d, were used for expression of the concatamer genes described above. These plasmids are part of the T₇ expression system that utilizes the T₇ phage RNA polymerase in conjunction with a T₇ promoter to drive recombinant gene expression (Studier, et al., Meth. Enzymol. 185:60-89 (1990)). For each of the concatamer genes, the gene fragment was released from the pUC118 plasmid by cleavage with Nco1 and BamH1 and purified. It was then cloned into the pET plasmid at the Nco1 and BamH1 sites placing the initiator ATG codon of the concatamer gene immediately adjacent to the T₇ promoter and ribosome binding sequences.

Escherichia coli is transformed with the plasmid by methods known in the art and the cells cultured. Expression of the concatamer genes was then analyzed by growing a bench-scale culture of the transformed E. coli and induction with or without the inducer isopropylthio-β-galactosidase (“IPTG”). Crude cell lysates of the cultures, taken pre- and post-induction, were electrophoresed on an SDS-PAGE and viewed following staining of the gels with CuCl₂. In addition, the proteins expressed from these small-scale cultures were purified following the procedures described below.

ii. Fermentation

Fermentations were performed using either Luria broth or Terrific Broth. Production of the protein-based polymers may also be induced with the addition of the inducing agent IPTG. After the appropriate number of hours of growth, the culture was harvested by centrifugation and cells disrupted by means of a French press to release the cell contents (Daniell, et al., Methods in Molecular Biology Series Entitled Expression and Detection of Recombinant Genes (1995)). The phenylalanine and lysine-containing polypeptide (or other such elastomer as the case may be) was then characterized for use.

3. Purification

The polymers were conveniently purified from culture lysates using their inverse temperature transitional properties (Urry, et al., Handbook of Biomaterials and Applications, Marcel Dekker, Inc., New York, pp. 2645-2699 (1995); Urry, Angew. Chem. (German) 105:859-883 (1993); Angew. Chem. Int. Ed. Engl. 32:819-841 (1993); and McPherson, et al., Protein Expression and Purification (1995)). The cell lysate was cooled to 4° C. and centrifuged at high speed to remove insoluble material. The bioelastomers form large aggregates and undergo phase separation (coacervation) when the supernatant fraction is either warmed to 37° C., or exposed to a high concentration of salt (NaCl) or subjected to a pH change, depending upon their composition. For lysine-containing bioelastomers such as Polymer XI, variance of the pH was used to achieve the differential phase separation that is key to the purification process.

The bioelastomer was then removed by centrifugation and solubilized under the appropriate conditions, (e.g., cooling or lowering salt concentration). Repeating this process several times provided a bioelastomer with essentially all other proteins removed. After centrifugation, the sample is dialyzed to remove any residual salts and then lyophilized to determine initial yield.

The solution was then to lower the temperature below its Tₜ so as to resolubilize 2% the coacervate followed by passing under pressure through a 0.2 micron membrane as a pre-filtration step and then through a sterile bench-top Amicon ProFlux M12 tangential flow ultrafiltration apparatus employing a 100 kDa spiral wound cartridge. The solution was then lyophilized.

B. Tyrosine-Containing Pentameric Bioelastic Materials

1. Gene construction

The gene constructions of Polymers IX′ and VII′ are given as examples of the primed series. The monomer gene encoding Polymer IX′, [(GVGIP)₁₁(GYGIP)]ₙ are constructed in three steps. Initially, the gene encoding for (GVGIP)lo is constructed and its sequence verified. Then the sequence for (GVGIP) (GYGIP) is made and attached to (GVGIP)₁₀. Finally, the whole unit, (GVGIP)₁₁(GYGIP) is cloned and sequenced again to verify the authenticity of the entire coding sequence.

2. Gene Expression and Fermentation

i. Expression

To construct (GVGIP)₁₀, two single-stranded oligonucleotides encoding the two halves of (GVGIP)₁₀ are chemically synthesized by a commercial source. The two opposing oligonucleotides then are annealed at their 3′ ends through a 20 base pair complimentary region, and extended to the full length, double stranded basic gene fragment by a high fidelity thermostable DNA polymerase. The resulting gene fragment is digested by the restriction enzyme BamHI before its insertion into the cloning vector pUC118 through ligation. After the transformation of the cloning host E coli DH5aF′, the positive clones are recovered from the selection plates and the plasmid DNA from each clone is isolated for the screening analysis. The resulting plasmids are digested with BamHI and separated on an agarose gel. The clones containing an insert of about 180 base pairs in length are the candidates for subsequent sequence verification. Once the sequence is verified, this clone is used as a source of (GVGIP)₁₀ for the ensuing gene construction. Similarly the two oligonucleotides encoding (GVGIP) (GYGIP) are also chemically synthesized. Upon annealing, this double stranded, full length DNA fragment is ligated to (GVGIP)₁₀ and cloned into vector pUC118. The possible clones are recovered from selection plates and the sequences of the positive clones are analyzed for bearing the correct sequence encoding monomer gene (GVGIP)₁₀(GVGIP) (GYGIP). This clone is then used as the source of monomer gene fragment for construction of concatemer genes.

The monomer gene encoding polymer VII′ (GVGIP)₂₁(GYGIP) is constructed in the similar three-step fashion. The resulting monomer gene has the sequence encoding (GVGIP)₁₁(GYGIP) (GVGIP)₁₀.

The construction of monomer genes encoding polymer XI′, XIII′, and XV′ (GVGIP)₂₁(GYGIP) are the same as for (GVGIP)₁₀. The resulting monomer genes have the sequences encoding (GVGIP)₈(GYGIP), (GVGIP)₅(GYGIP), and [(GVGIP)₂(GYGIP)]₃, respectively.

To construct the concatemer (multimer) gene, a large amount of the monomer gene fragments are prepared after digesting the monomer gene containing plasmid with the restriction enzyme. The resulting monomer gene fragments are concatenated (ligated) in the presence of N- and C-terminal adaptors, which provide the cloning sites for the subsequent manipulation in different vectors. The resulting concatenation products, consisting of multimer genes of varying chain length, are ligated into pUC118 and introduced into E coli. The possible clones on the selection plates are screened and the positive clones identified after a series of digestion with different restriction enzymes.

ii. Fermentation

Fermentations were performed using either Luria broth or Terrific Broth. Production of the protein-based polymers may also be induced with the addition of the inducing agent IPTG. After the appropriate number of hours of growth, the culture was harvested by centrifugation and cells disrupted by means of a French press to release the cell contents (Daniell, et al., Methods in Molecular Biology Series Entitled Expression and Detection of Recombinant Genes (1995)). The phenylalanine and lysine-containing polypeptide (or other such elastomer as the case may be) was then characterized for use.

3. Purification

The polymers were conveniently purified from culture lysates using their inverse temperature transitional properties (Urry, et al., Handbook of Biomaterials and Applications, Marcel Dekker, Inc., New York, pp. 2645-2699 (1995); Urry, Angew. Chem. (German) 105:859-883 (1993); Angew. Chem. Int. Ed. Engl. 32:819-841 (1993); and McPherson, et al., Protein Expression and Purification (1995)). The cell lysate was cooled to 4° C. and centrifuged at high speed to remove insoluble material. The bioelastomers form large aggregates and undergo phase separation (coacervation) when the supernatant fraction is either warmed to 37° C., or exposed to a high concentration of salt (NaCl) or subjected to a pH change, depending upon their composition. For lysine-containing bioelastomers such as Polymer XI, variance of the pH was used to achieve the differential phase separation that is key to the purification process.

The bioelastomer was then removed by centrifugation and solubilized under the appropriate conditions, (e.g., cooling or lowering salt concentration). Repeating this process several times provided a bioelastomer with essentially all other proteins removed. After centrifugation, the sample is dialyzed to remove any residual salts and then lyophilized to determine initial yield.

The solution was then to lower the temperature below its Tₜ so as to resolubilize the coacervate followed by passing under pressure through a 0.2 micron membrane as a pre-filtration step and then through a sterile bench-top Amicon ProFlux M12 tangential flow ultrafiltration apparatus employing a 100 kDa spiral wound cartridge. The solution was then lyophilized.

### Example 2

### Forming Bioelastomers with Incorporated Cations

A variety of bioelastomers, produced in the form of sheets, were evaluated as to their absorption characteristics:

Polymer I: (GVGVP)₂₅₁ (SEQ ID NO: 6, where X¹ is V and X² is V and n=251), γ-radiation (6, 10, 14, 18, 20, 22, 26 and 30 Mrad) cross-linked.

Polymer II: (GVGIP)₂₆₀ (SEQ ID NO: 6, where X¹ is V and X² is I and n=260) γ-radiation (6, 10, 14, 18, 22, 26 and 30 Mrad) cross-linked.

Polymer VIII: (GVGIP GFGEP GEGFP GVGVP GFGFP GFGIP GVGIP GVGIP GFGEP GEGFP GVGVP GFGFP GFGIP)ₙ(GVGVP), where n is 20 (SEQ ID NO: 13).

Polymer I, a GVGVP-based bioelastomer, was synthesized as described above in Example 1 and was used as a control. Polymers III and VIII were made in a manner similar to Example 1, with the exception that they were made with a COO⁻ anion site, with Na⁺ or Ca⁺² titrated in as cations to form the ion-pair dipole. Polymer III was a modified GVGVP-based bioelastomer, having one glutamic acid and five phenylalanine residues in each 30 amino acid unit. Polymer VIII was a modified GVGVP-based bioelastomer, having four glutamic acid, ten phenylalanine and four isoleucine residues in each sixty-amino acid unit.

Acoustical Measurements

Direct acoustical transmission measurements were taken to determine the absorption spectra of the sample polymers, and to identify any strong absorption dips. An experimental set-up was developed to measure transmitted acoustical waves though the sample polymers. The sample polymer was placed within a vessel containing a salt (NaCl) solution. The vessel was positioned on an accelerometer, which lead to a spectrum analyzer. The sample polymer was positioned between the accelerometer and a hydrophone, which was also positioned within the vessel.

Measurements were taken to determine the acoustical spectra of the salt solution. Polymer I, Polymer III and Polymer VIII. The spectra of Polymers III and VIII showed additional absorption valleys than those observed with the salt solution and with Polymer I.

The spectral data from Polymers III and VIII were therefore subtracted from those of the salt solution. The difference spectra showed deep absorption valley at 70 kHz for Polymer III and at 50 kHz for Polymer VIII. The spectral data from Polymers III and VIII were also subtracted from those of Polymer I. Similar absorption valleys were observed.

It was concluded that the two cation-modified bioelastomers, Polymer III and VIII showed deep absorption valleys at 70 kHz and 50 kHz, respectively.

Elastic Parameters

All of the main elastic parameters of the sample polymers were measured: complex shear modulus, Poisson ratio, loss factor and compression modulus. The loss factor is approximately equal to the absorption coefficient measured above.

The experimental set-up utilized a shaker, impedance head, accelerometer and a network analyzer to determine the measured values for the transfer matrix. The transfer matrix was then inverted offline to obtain the elastic parameters.

### Example 3

### Introduction of Magnesium Sulfate Function

Additional genes can be designed to introduce the magnesium sulfate function. It is the ion-pair dissociation of magnesium sulfate in seawater that exhibits a high acoustic absorption that is far beyond its relative concentration.

In order to evaluate an organic magnesium sulfate site for acoustic absorption, poly[fᵥ(GVGVP),f_{y}(GY(SO₃)GVP)] (SEQ ID NO: 28), where “Y” represents a tyrosine residue, was used At room temperature, there was limited absorption. However, upon raising the temperature to 40° C., strong absorption was observed. This warranted the production of a tyrosine-containing, more hydrophobic polymer.

The gene construction of tyrosine-containing copies of (GVGIP)lo (SEQ ID NO: 6, where X¹ is V and X² is I and n=10) is based upon basic monomer genes containing tyrosine to GVGIP rations of 1:51 or 1:101. The tyrosine residues replace the V of GVGIP, and are well suited as sites for sulfation. Once sulfated, these sulfate-containing polymers are then ion-paired with magnesium and evaluated for low frequency acoustic absorption and dielectric relaxation characteristics.

### Example 4

### Relaxation Frequencies of Poly(VPGVG)-Type Polymers

In particular, low frequency dielectric relaxation studies have recently shown that the repeating unit (the β-turn-molecular spring) can be tuned to absorb, i.e., to resonate, at frequencies below 100 kHz. Specifically, Polymer I (SEQ ID NO: 6, where X¹ is V and X² is V and n=251) with the (GVGVP) unit (having a pentamer repeat weight of 409 Da) resonates with a broad band centered at 100 kHz; Polymer II (SEQ ID NO: 6, where X¹ is V and X² is I and n=260) with the (GVGIP) unit, which is a larger mass unit with a pentamer weight of 423 Da, resonates with a broad band at 23 kHz, and Polymer III (SEQ ID NO: 8) with a mean pentamer repeat weight of 454 Da resonates with a broad band centered at 3 kHz.

The polymers were characterized in the form of 20 Mrad cross-linked sheets, and temperatures were 25° C. for Polymers II and III and 40° C. for Polymer I in order to be above the inverse temperature transition for hydrophobic folding and assembly. Below the temperature of the inverse temperature transition of hydrophobic folding and assembly, these intense peaks were not observed.

### Example 5

### Relaxation Frequency as a Function of Mass of Repeating Unit

As seen in FIG. 3, a plot of 1n frequency versus mean pentamer weight gives a straight line with a slope of −0.07. This data includes a temperature correction for Polymer I, which slightly lowered its resonance frequency and the slope from −0.08 to −0.07 and provided an even better straight line. Thus, the frequency of the dielectric relaxation appears to be inversely proportional to mass of the mean repeating unit, as might be expected for the mechanical process of acoustic absorption.

As can be seen in FIGS. 1A to 1D, the β-turn acoustic vane for Polymer III would resonate with a broad band peaking at 3 kHz (see Example 4, which describes the relationship between dielectric relaxation and acoustic absorption), and the absorbed energy would readily dissipate into the water column within the β-spiral and the water surrounding the suspended segments.

The data for Polymers I, II and III demonstrates a capacity to design for the desirable range. Moving to lower frequencies, the slope of FIG. 4 would predict a band centered near 1.2 kHz for Polymer VIII with a mean pentamer repeat weight of 464 Da. Finally, it might be noted that a relaxation at submegaherz frequencies had been suggested on the basis of the analysis of the real part of the dielectric permitivity in the 1 MHz to 1 GHz range for Polymers I and II (See Buchet, et al., J. Phys. Chem. 92:511-517, 1988). The range of interest for the uses described herein is 40 Hz to 40 kHz, or more specifically from 200 Hz to 7 kHz. The capacity of bioclastomers to absorb in this range has been remarkably demonstrated by the loss factor curves of FIGS. 2 and 3.

### Example 6

### Varying Cross-Link Density to Lower Frequency

In addition to increasing repeat molecular weight as a means of lowering the relaxation frequency, both increasing cross-link density and changing from aqueous toward non-aqueous media can be used to lower the relaxation frequency. Preliminary characterizations of the temperature dependence of a chemically cross-linked sample of Polymer II indicate dielectric relaxation frequencies ranging from several hundred Hz to 5 kHz on raising the temperature from 7° C. to 50° C., as shown in FIG. 5. Thus, it would seem that the frequency range of interest could be blanketed by controlling cross-link density and with capacity to plan for the ambient temperature ranges during use.

### Example 7

### Coupling to Ion-Pair Dissociation to Tune Frequency

It is believed that the mechanism of ion-pair dissociation under the influence of a variable hydrophobic environment can be used to tune toward the desired frequency range. This is supported by findings in seawater where sound induced dissociation of MgSO₄ and of borate salts contribute to substantial acoustic absorption at 1 kHz and below. The latter capacity is readily designed into the protein-based polymers described herein. It is expected that increased hydrophobicity of the protein-based polymer will lower the frequency for the ion-pair dissociation/association.

As described in Example 3, by utilizing a tyrosine as a sulfation site and varying the fraction of pentamers that contain the site, it becomes possible to vary the mean pentamer mass in addition to increasing the capacity to utilize the ion-pair dissociation. Using the slope of FIG. 4 and the composition poly[(IPGVG)ₓ(IPGY{SO₄}G)] to give the primed polymers (SEQ ID NOS: 49 through 58), an x of 1 would give 100 Hz, and an x of 10 would give 9.2 kHz. If one of the values of x gives a frequency that matches the rate of ion-pair dissociation, then a particularly intense acoustic absorption might be anticipated.

What appears evident is that there are many ways to design for acoustic absorption in a desired frequency range, particularly when using elastic protein-based polymers with librational motional modes available.

### Example 8

### Elastic Protein-Based Polymers Designable for Desired Shear Modulus

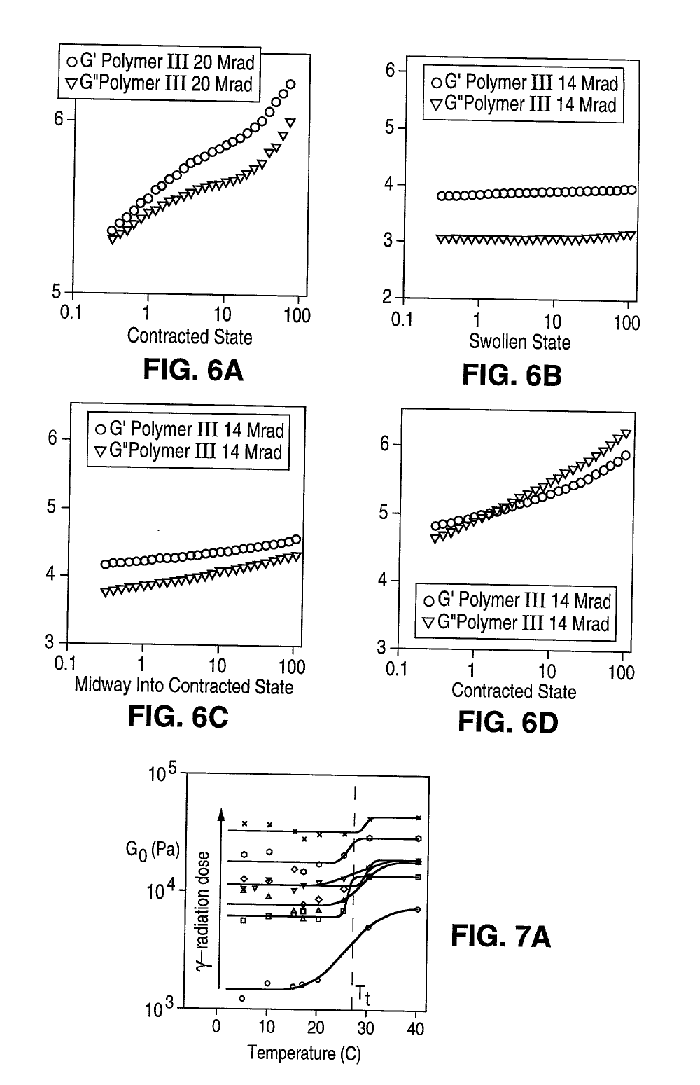

Not only does it appear possible to design for the desired frequency range, but it is also possible to design for desired shear moduli. For example, Polymer III has been observed with shear moduli ranging from 10⁵ Pa at 0.1 rad/s to greater than 10⁶ Pa at 100 rad/s, as shown in FIG. 6A. This is not characteristic of elastomers composed of random chain networks where G′ and G″ remain more constant with increasing test frequency, and it is not characteristic of the elastic protein-based polymer at conditions below the transition temperature, before hydrophobic folding and assembly, as shown in FIG. 6B. For a 14 Mrad cross-linked sample, as the pH is lowered from 8.1 to 7.6 and on to 6.5, i.e., toward the pKa of 6.6 for hydrophobic folding and assembly of the uncross-linked polymer, the shear moduli progressively increase with increasing test frequency (See FIGS. 6B, 6C and 6D). Accordingly, with these regular, non-random polypeptide elastomers, the shear moduli appear to increase as the frequency approaches the relaxation frequency of the dielectric relaxation. It may also be seen on comparing FIGS. 6A and 6D that the shear moduli increase with increase in cross-linking by gamma irradiation.

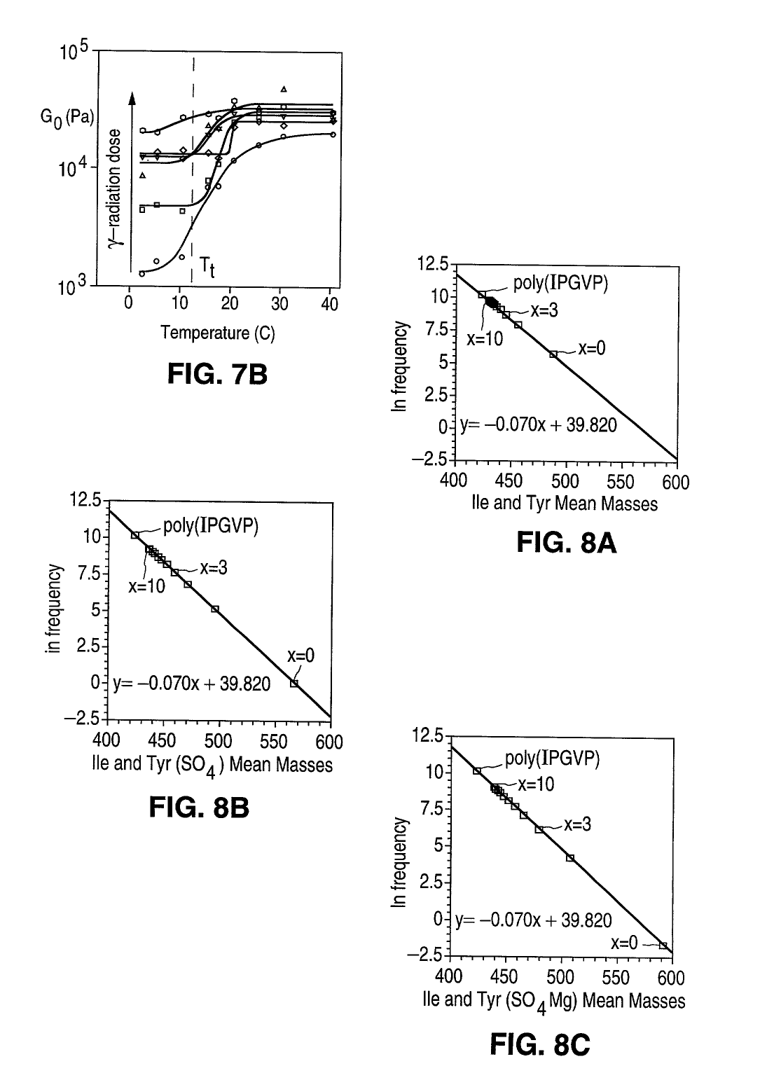

Furthermore, by increasing the cross-link density, these polymers can be converted from hydrogels through the range of elastomers with increasing shear moduli to the stage of what could be called hard plastics. Experimentally, shear moduli increase on raising the temperature from below to above the transition temperature range and with increase in cross-link density, e.g., with increase in Mrad cross-linking dose (See FIGS. 7A-B). For weakly (10 Mrad) cross-linked samples of Polymers I and II, the shear moduli are also seen to increase on going from below to above the temperature range of the inverse temperature transition and the slope as a function of test frequency is greater above the transition. Related changes are found in the low frequency dielectric relaxation data.

Clearly, the dependencies of the relevant physical properties are more complex for elastic protein-based polymers than for elastomers based on random chain networks. It is for this reason that elastic protein-based polymers exhibit greater promise for maximizing acoustic absorption over frequency ranges of interest.

### Example 9

### Dielectric Relaxation and the Damping Vibration of the β-Turn Acoustic Vane

The Measurement: The dielectric relaxation experiment, in its simplest embodiment used for the low frequencies of direct interest here, constitutes a pair of spaced parallel plates, one positively charged and the other negatively charged, where the charges on the plates can be reversed at frequencies that can range from less than 1 Hz to as high as 26.5 GHz (Approaching the latter high frequencies requires different instrumental approaches). Molecules placed between the two plates will provide a high dielectric constant if they contain orientable dipole moments such as with water or with a carbonyl, C═O, moiety. A dipole moment within a molecule will attempt to orient pointing its negative end (for example the oxygen atom of the carbonyl) toward the positive plate and its positive end (the carbon atom) toward the negative plate, and to change its orientation at the frequency of plate charge reversal.

Random Chain Molecules: For long chain molecules the frequency at which the dipole moment can reorient depends on the energy barriers for changes in the torsion angles attending, for example, the C═O moiety. In random chain networks, because the chain does not form a regular structure, the barrier for rotation about particular bonds will differ because each chain and each segment along a chain assumes different configurations. Accordingly, motional modes due to regular structures are not expected to exist as the energy barriers for rotation about given torsion angles are expected to be different from one site to the next in the random chain networks ascribed to classical rubbers.

Regularly Structured Repeating Peptide Units: If on the other hand the chain contains repeating peptide units with preferred sets of torsion angles, then the energetics, the barrier for rotation about given torsion angles, can be the same from one repeat to the next. For poly(VPGVG) the only dipole moments occur in the peptide moiety, CONH, with the carbonyl dipole moment component being dominant. In fact as reviewed below, intense relaxations can occur at the frequency that reflects the energy barrier for the collective motion of the peptide moieties of the repeating unit.

Specifically, an intense relaxation near 10 MHz with a dielectric increment of 70 has been observed in the dielectric relaxation spectrum of 400 mg/ml concentrations of poly(GVGVP) in water at 40° C. This intense relaxation is not found at 25° C.; it develops on raising the temperature to form the hydrophobically folded β-spiral structure described above.

At this polymer concentration, the concentration of peptide moieties is about 5 molar, one-tenth that of pure water, and yet it gives a dielectric increment that is as great as the entire dielectric constant for 55 molar water. This argues for repeating pentamer units exhibiting the same frequency for their collective dipole moment re-orientations.

Relationship of the Summed Peptide Dipolar Reorientations of the Pentamer Unit to the Intensity of a 10 MHz Dielectric Relaxation: From the temperature dependence of the correlation time above 40° C. for poly(VPGVG), the barrier for the peptide dipole re-orientation is 1.2 kcal/mole. Accordingly, the family of conformations of the pentamer unit with energies within 1 to 1.5 kcal/mole of the lowest energy can provide the correlated set of dipole moment changes for the five peptide moieties of the repeating pentamer of poly(VPGVG). The individual five dipole moment changes are summed to give the resultant dipole moment change for the pentamer as a whole. Using Onsager's relation for polar liquids, this dipole moment change resulting from the collective motion of the pentamer is used to calculate the expected dielectric increment. The calculated value fits the experimental value within a factor of two when using the 1.5 kcal barrier and within 10% for the 1 kcal barrier height. Furthermore, the ratio of values between the (GVGVP) pentamer and an analog, (GVAVP) with one-third the intensity of relaxation, agrees essentially exactly. (See Venkatachalam, et al., Int. J. Quant. Chem.: Quant. Biol. Symp. 12:15-24, 1986). Thus, there is substantial reason to think of pentamer motional modes resulting from the correlated motions of the five constituent peptides of the pentamer.

### Example 10

### Coupling of Ion-Pair Dissociation Frequency to the Molecular Springs Connecting β-Turns

As a means of further tuning and enhancing the intensity of the acoustic absorption, an additional element involving ion-pair dissociation can be incorporated into the polymer design to couple to the fundamental acoustic absorbing unit. As noted above, seawater exhibits a substantial acoustic absorption near the 1 kHz frequency range and below due to an acoustically driven dissociation/association of MgSO₄ and borate salt ion-pairs. A polypentapeptide sequence has been designed, within which a sulfate can be attached to a residue that replaces the V of GVGIP. The magnesium sulfate salt will then be formed (which in itself increases mass), and it is expected that the controllable increase in hydrophobicity of the polymer will result in a decrease of the frequency of the acoustically-driven ion-pair dissociation.

Using the slope of FIG. 4 and the composition poly[(IPGVG)ₓ(IPGY{SO₄}G)], with x ranging from 0 to 10 the set of frequencies would be expected as shown in FIGS. 7A, B and C for Tyr(Y)-containing, sulfated-Tyr-containing and for magnesium sulfate-Tyr-containing polymers, respectively. The frequency in the plot of FIG. 8C at which the frequency of the librational motional mode became matched by the frequency of the magnesium sulfate dissociation/association within the elastic protein-based polymer matrix could be expected to result in an enhanced absorption intensity in the desired frequency range.

### Example 11

### Protein-Based Acoustic Absorbers as Transductional Elements

Because the dielectric relaxation is emerging as an effective reporter of the motion that results in acoustic absorption, it is expected to make an interesting monitor or sensor of the actuation of the protein-based polymer by an acoustic wave. Thus, a compressional wave striking a sample of properly designed elastic protein-based polymer placed between a pair of parallel plates should become transducible into an electrically detectable dielectric relaxation, that is, into a change in dielectric permitivity of the sample between the parallel plates of the dielectric relaxation instrument.

All publications and patent applications mentioned in this specification are herein incorporated by reference to the same extent as if each individual publication or patent application was specifically and individually indicated to be incorporated by reference at the location where cited.

The invention now being fully described, it will be apparent to one of ordinary skill in the art that many changes and modifications can be made thereto without departing from the spirit or scope of the invention.

What is claimed is:

1. A method for reducing the acoustical noise, reducing the sonar cross-section or reducing the radar cross-section of an object, comprising covering the object with a polymer which comprises repeating peptide monomeric units selected from the group consisting of nonapeptide, pentapeptide and tetrapeptide monomeric units, wherein said monomeric units form a series of β-turns separated by dynamic bridging segments suspended between said β-turns.

2. The method of claim 1 wherein said polymer is cross-linked.

3. The method of claim 1 wherein said polymer comprises a block or random copolymer comprising at least two of said monomeric units.

4. The method of claim 1 wherein said polymer comprises an elastomeric polytetrapeptide or polypentapeptide.

5. The method of claim 1 wherein said polymer is comprised of pentapeptide monomeric units selected from the group consisting of GVGVP (SEQ ID NO: 6, where X¹ is V and X² is V), GVGIP (SEQ ID NO: 6, where X¹ is V and X² is I), GVGFP (SEQ ID NO: 6, where X¹ is V and X² is F), GFGFP (SEQ ID NO: 6, where X¹ is F and X² is F), GFGEP (SEQ ID NO: 6, where X¹ is F and X² is E), GFGIP (SEQ ID NO: 6, where X¹ is F and X² is I), GEGFP (SEQ ID NO: 6, where X¹ is E and X² is F), GEGVP (SEQ ID NO: 6, where X¹ is E and X² is V), GKGFP (SEQ ID NO:6, where X¹ is K and X² is F), GKGVP (SEQ ID NO:6, where X¹ is K and X² is V), GEGIP (SEQ ID NO: 6, where X¹ is E and X² is I), GKGIP (SEQ ID NO: 6, where X¹ is K and X² is I) and GYGIP (SEQ ID NO: 6, where X¹ is Y and X² is I), alone or in combination.

6. The method of claim 5 wherein at least one of said pentapeptide monomeric units is GVGVP (SEQ ID NO:6, where X¹ is V and X² is V) or GVGIP (SEQ ID NO:6, where X¹ is V and X² is I).

7. The method of claim 1 wherein said polymer comprises at least one pentapeptide monomeric unit having the formula GX¹GX²P (SEQ ID NO:6), where X¹ is selected from the group consisting of V, E, F, Y and K; and X² is selected from the group consisting of V, E, F and I.

8. The method of claim 1 wherein said polymer comprises at least monomeric unit containing a phenylalanine, tyrosine or isoleucine residue.

9. The method of claim 1 wherein said polymer is modified to contain at least one hydrophobically tuned ion-pair dissociable site.

10. The method of claim 9 wherein said polymer comprises at least one pentapeptide monomeric unit having the formula GX⁴GX⁵P (SEQ ID NO:24) where X⁴ is selected from the group consisting of V, E, F, Y, K, S and T; and X⁵ is selected from the group consisting of V, E, F, I, S, T and Y; with the proviso that at least one of X⁴ and X⁵ is Y, S, or T.

11. The method of claim 9 wherein said polymer comprises at least one monomeric unit having the formula -GVGVP-X³-GVGVP- (SEQ ID NO:23) where X³ in is S, T or Y.

12. The method of claim 1 wherein said polymer absorbs frequencies within the range of 100 Hz to 100 kHz.

13. The method of claim 1 wherein the entire monomeric unit or β-turn and suspended segment exhibits a collective motional mode in the low frequency range below 100 kHz.

14. The method of claim 1 wherein the object is a submarine or ship.

15. The method of claim 14 wherein said covering comprises coating the hull of the submarine or ship with said polymer.

16. The method of claim 15 which further comprises covering said polymer coating with a protective non-frequency reflecting coating.

17. A method for reducing the acoustical noise, reducing the sonar cross-section or reducing the radar cross-section of an object comprising covering the object with an amphiphilic petroleum-based polymer, which exhibits lower critical solution temperature behavior and contains at least one hydrophobically tuned ion-pair dissociable site.

18. The method of claim 17 wherein said polymer absorbs frequencies within the range of 100 Hz to 100 kHz.

19. The method of claim 17 wherein the object is a submarine or ship.

20. The method of claim 19 wherein said covering comprises coating the hull of the submarine or ship with said polymer.

21. The method of claim 20 which further comprises covering said polymer coating with a protective non-frequency reflecting coating.

22. The method of claim 21 wherein said polymer is selected from the group consisting of acrylamides and esters.

23. A method for measuring the sound absorption capabilities of a protein-based material comprising the steps of: (a) forming a test component from a polymer comprising repeating peptide monomeric units selected from the group consisting of nonapeptide, pentapeptide and tetrapeptide monomeric units, wherein said monomeric units form a series of β-turns separated by dynamic bridging segments suspended between said β-turns, wherein the polymer is optionally modified to include a charged site; (b) exposing the test component to a high intensity, low frequency sound; (c) measuring the change in dielectric increment of the test component; and (d) correlating the measured dielectric increment of the test component to the level of sound that would be absorbed by a protein-based material when exposed to the high intensity, low frequency sound.

24. The method of claim 23 wherein said protein-based material is marine mammal tissue or other biological tissue.

25. A method of measuring the microwave or radar absorption capabilities of a material comprising the steps of: (a) forming a test component from a polymer comprising repeating peptide monomeric units selected from the group consisting of nonapeptide, pentapeptide and tetrapeptide monomeric units, wherein said monomeric units form a series of β-turns separated by dynamic bridging segments suspended between said β-turns, wherein the polymer is optionally modified to include a charged site; (b) exposing the test component to microwave or radar exposure; (c) measuring the change in dielectric increment of the test component; and (d) correlating the dielectric relaxation to the amount of microwave or radar absorption that would occur when the material is exposed to microwaves or radar.

26. The method of claim 25 wherein the method measures the radar absorption capabilities of a protein-based material.

27. The method of claim 25 wherein the method measures the microwave absorption capabilities of a polymers with hydrophobic hydration.

28. A method of designing polymers that are capable of low frequency acoustic absorption, comprising the steps of: (a) forming a polymer comprised of repeating peptide monomeric units selected from the group consisting of nonapeptide, pentapeptide and tetrapeptide monomeric units, wherein said monomeric units form a series of β-turns separated by dynamic bridging segments suspended between said β-turns; and (b) introducing a charged site on said polymer.

29. The method of claim 28 wherein said charged site is an anionic site.

30. The method of claim 28 wherein said charged site is a cationic site.

31. The method of claim 28 wherein said polymer is cross-linked.

32. The method of claim 28 wherein said polymer comprises a block or random copolymer comprising at least two of said monomeric units.

33. The method of claim 28 wherein said polymer comprises an elastomeric polytetrapeptide or polypentapeptide.

34. The method of claim 28 wherein said polymer is comprised of pentapeptide monomeric units selected from the group consisting of GVGVP (SEQ ID NO:6, where X¹ is V and X² is V), GVGIP (SEQ ID NO: 6, where X¹ is V and X² is I), GVGFP (SEQ ID NO:6, where X¹ is V and x² is F), GFGFP (SEQ ID NO:6, where X¹ is F and X² is F), GFGEP (SEQ ID NO: 6, where X¹ is F and X² is E), GFGIP (SEQ ID NO: 6, where X¹ is F and X² is I), GEGFP (SEQ ID NO:6, where X¹ is E and X² is F), GEGVP (SEQ ID NO: 6, where X¹ is E and X² is V), GKGFP (SEQ ID NO: 6, where X¹ is K and X² is F), GKGVP (SEQ ID NO:6, where X¹ is K and X² is V), GEGIP (SEQ ID NO: 6, where X¹ is E and X² is I), GKGIP (SEQ ID NO: 6, where X¹ is K and X² is I) and GYGIP (SEQ ID NO: 6, where X¹ is Y and X² is I), alone or in combination.

35. The method of claim 34 wherein at least one of said pentapeptide monomeric units is GVGVP (SEQ ID NO: 6, where X¹ is V and X² is V) or GVGIP (SEQ ID NO: 6, where X¹ is V and X² is I).

36. The method of claim 28 wherein said polymer comprises at least one pentapeptide monomeric unit having the formula GX¹GX²P (SEQ ID NO: 6), where X¹ is selected from the group consisting of V, E, F, Y and K; and X² is selected from the group consisting of V, E, F and I.

37. The method of claim 36 wherein at least one of X¹ and X² is phenylalanine.

38. The method of claim 28 wherein said polymer comprises at least monomeric unit containing a phenylalanine, tyrosine or isoleucine residue.

39. The method of claim 28 wherein said polymer contains at least one residue selected from the group consisting of serine, threonine and tyrosine.

40. The method of claim 39 wherein said residue is contained within a monomeric unit.

41. The method of claim 39 wherein said residue is between two monomeric units.

42. The method of claim 28 wherein said polymer absorbs frequencies within the range of 1100 Hz to 100 kHz.

43. The method of claim 28 wherein said introduction of a charged site on the polymer allows for subsequent formation of an ion-pair dipole.

44. The method of claim 43 wherein the cation in said ion-pair dipole is selected from the group consisting of alkali, alkaline earth, transition metal ions, lanthamides and actinides.

45. The method of claim 44 wherein said cation is selected from the group consisting of Na⁺, Ca⁺, Mg⁺², Ba⁺², Sr⁺² and Pb⁺².

46. The method of claim 43 wherein the anion in said ion-pair dipole is selected from the group consisting of carboxylates, phosphates, sulfates, borates and silicates.

47. The method of claim 46 wherein said anion is selected from the group consisting of —COO⁻, —OSO₃⁻², OPO₃⁻² and —OBO₂⁻².

48. A method of designing polymers that are capable of low frequency acoustic absorption, which comprises forming a polymer comprised of repeating peptide monomeric units selected from the group consisting of nonapeptide, pentapeptide and tetrapeptide monomeric units, wherein said monomeric units form a series of β-turns separated by dynamic bridging segments suspended between said β-turns, wherein said polymer has one or more of the following characteristics: a mean mass of the repeating unit within the range of 300 to 10,000 daltons; a cross-link density of the matrix within the range of one cross-link per 300 dalton to no cross-links between chains (coacervate state); a water content of the matrix or coacervate within the range of 2% to 99% by weight; a hydrophobicity of the repeating unit within the Tₜ range of −200° C. to 90° C.; and a transition temperature within the range of −200° C. to 120° C.

49. A protein based polymer for use in reducing acoustical noise, reducing sonar cross-section or reducing radar cross-section comprising a polymer having repeating units and at least one of the following characteristics: a mean mass of the repeating unit within the range of 300 to 10,000 daltons; a cross-link density of the matrix within the range of one cross-link per 300 dalton to no cross-links between chains (coacervate state); a water content of the matrix or coacervate within the range of 2% to 99% by weight; a hydrophobicity of the repeating unit within the Tₜ range of −200° C. to 90° C.; and a transition temperature within the range of −200° C. to 120° C.

50. The protein based polymer of claim 49 wherein said polymer is selected from the group consisting of the protein formulas of SEQ ID NO:6, where X¹ is V and X² is V and n=25 1; SEQ ID NO:6, where X¹ is V and X² is I and n=260; SEQ ID NOS:8-20 and SEQ ID NOS:29-44.

51. A kit for reducing the acoustical noise, sonar cross-section or radar cross-section of an object, comprising: (a) a polymer comprised of repeating peptide monomeric units selected from the group consisting of nonapeptide, pentapeptide and tetrapeptide monomeric units, wherein said monomeric units form a series of β-turns separated by dynamic bridging segments suspended between said β-turns, wherein said polymer has one or more of the following characteristics: a mean mass of the repeating unit within the range of 300 to 10,000 daltons; a cross-link density of the matrix within the range of one cross-link per 300 dalton to no cross-links between chains (coacervate state); a water content of the matrix or coacervate within the range of 2% to 99% by weight; a hydrophobicity of the repeating unit within the Tₜ range of −200° C. to 90° C.; and a transition temperature within the range of −200° C. to 120° C.; and (b) a material to form a protective coating.

52. The kit of claim 51 which further comprises a cross linking agent.
